(19) United States
(12) Patent Application Publication
(10) Pub. No.: US 2026/0337253 A1
(43) Pub. Date: Oct. 29, 2026

(54) INHIBITION OF GUT BACTERIA

(71) Applicant: THE BOARD OF TRUSTEES OF THE UNIVERSITY OF ILLINOIS, Urbana, IL (US)

(72) Inventors: Wilfred A. VAN DER DONK, Champaign, IL (US); Bidisha CHAKRABORTY, Urbana, IL (US); Ryan MOREIRA, Champaign, IL (US); Satish NAIR, Champaign, IL (US)

(21) Appl. No.: 19/633,622

(22) Filed: Mar. 30, 2026

Related U.S. Application Data
(60) Provisional application No. 63/779,530, filed on Mar. 28, 2025.

(51) Int. Cl. C07K 14/81; A61K 38/00; A61P 31/04

(52) U.S. Cl. CPC C07K 14/811; A61P 31/04; A61K 38/00

(57) ABSTRACT

The disclosure provides for certain peptide-based enzymatic inhibitors of Enterococcus species, and in particular, inhibitors of the CylA serine protease of Enterococcus faecalis and methods of using the same to treat an Enterococcus faecalis infection.

### RELATED APPLICATIONS

This application claims priority under 35 U.S.C. § 119(e) to U.S. Provisional Patent Application No. 63/779,530, filed Mar. 28, 2025, which is incorporated herein by reference.

### GOVERNMENT SUPPORT

This invention was made with government support under R37 GM058822 awarded by the National Institutes of Health. The United States government has certain rights in the invention.

### REFERENCE TO SEQUENCE LISTING

The present application includes a Sequence Listing in electronic format as an xml file titled “500.168US1_SL” which was created on Mar. 30, 2026, and has a size of 18,265 bytes. The contents of xml file 500.168US1_SL are incorporated herein by reference.

### BACKGROUND OF THE INVENTION

Natural products have had a remarkable impact on human health and many therapeutics are based on natural product scaffolds. Notable examples include penicillin, streptomycin, daptomycin, and bleomycin all of which are mainstays in the clinical arsenal for treating life threatening infections and cancers. In addition, understanding of how natural products produced by the microbiome affect human health has advanced considerably in recent years.

The human microbiome consists of trillions of organisms colonising several organ systems throughout the human body. This collection of organisms is responsible for healthy nervous system development and plays important roles in digestion and immunity. Members of the human microbiome communicate with their host and with each other through a number of mechanisms including the production and detection of small molecules. In some cases, these small molecules can significantly impact human health. For example, colibactin, a natural product produced by Escherichia coli, is a genotoxic prodrug that alkylates DNA and has been implicated in colon-rectal cancers. Conversely, lugdunin, a cyclic peptide natural product produced by Staphylococcus lugdunesis, mobilizes the human immune system against opportunistic Staphylococcus aureus, reducing the incidence and severity of skin infections.

Ribosomally synthesized and post translationally modified peptides (RiPPs) are a class of natural products in which the substrate sequences are genetically encoded and often clustered together with the genes for the modification enzymes. Lanthipeptides are a subclass of RiPPs that have a wide array of bioactivities ranging from antibacterial to antiviral to morphogenic. When a lanthipeptide has antibiotic properties, it is often referred to as a lantibiotic. The best known lantibiotic is nisin, which has activity against a large panel of Gram-positive organisms and has been used for decades as a food preservative against food-borne pathogens. Recently, it was discovered that nisin-like lantibiotics are produced by members of the human gut microbiome, and that the presence of these lantibiotics in the gut microbiome decreases the likelihood of dense colonization of the intestinal tract by vancomycin resistant enterococci (VRE). Another lantibiotic, salivaricin, is excreted by commensal organisms found in the mouth and affects the biodiversity of the oral microbiome through immunomodulatory stimulation and selective antimicrobial activity. Conversely, other lanthipeptides produced by members of the human microbiome, such as the enterococcal cytolysin, have the potential to negatively impact human health through complicating existing disease states.

Despite limited details describing the interaction of CylA with its self-cleavage or substrate sequence, existing models suggest that these sequences may serve as starting points for the development of CylA inhibitors, however, no effective CylA inhibitors currently exist. Accordingly, there is a need for a new and effective CylA inhibitors to treat or prevent certain Enterococcus Sp. infections. The present disclosure satisfies these needs.

### SUMMARY OF THE INVENTION

Applicants provide a series of peptide boronic acid inhibitors of CylA based on its natural recognition sequences was developed and this collection of inhibitors was used to gain insights into the interaction of CylA with peptidic substrates. In vitro studies demonstrated that peptide boronic acids with sequences similar to the substrate or self-cleavage sequence inhibited the proteolytic activity of CylA and blocked the conversion of mCylL_{L} or mCylL_{S} to their mature and toxic counterparts. Studies on enzyme-inhibitor interactions revealed that CylA undergoes activation in a substrate-dependent manner and that inhibitor binding facilitates irreversible release of the pro-domain from the catalytic C-terminal domain. An X-ray structure of CylA non-covalently bound to its pro-domain provides a high-resolution glimpse into substrate-enzyme interactions and provides additional insights into the inhibitor-enzyme structure-activity relationship. Finally, experiments on wild type C+ E. faecalis demonstrated that these inhibitors attenuated the hemolytic ability of cytolysin producers by halting the maturation of cytolysin and the expression of the cytolysin BGC.

Accordingly, in some embodiments, a peptide comprises, from an N-terminus to a C-terminus, the Formula I:

Z-X¹-X²-X³-X⁴-X⁵-X⁶-BA (I),

wherein Z is an acetyl group, a thiazole group, an imidazole group, a pyrazine group, a difluorophenyl group, a dichlorophenyl group, or absent, wherein Z, when present, is covalently bonded to an N-terminus nitrogen on X¹; X¹ is Gly, Pro, Ser, cyclopropylglycine (Cpg), cyclopentylglycine (Cpg*), Asp, Gln, or Ala; X² is Asp, Glu, Gln, Ala, Phe, Ile, Cpg, Norvaline (Nva), or 2-aminobutyric acid (Abu); X³ is Lys, Gln, Val, Phe, Cpg, Ala, Pro, Nva, or absent; X⁴ is Glu, Gln, Lys, Pro, Ala, Nva, or absent; X⁵ is Ala, Pro, Nva, Abu, or absent; X⁶ is Gly, Glu or Nva; and BA is a boronic acid moiety (—B(OH)₂) covalently bonded to a carbon atom of an amino acid at the C-terminus of the peptide (i.e., X⁶) and/or the BA moiety replaces the —(COOH) of the C-terminal amino acid moiety (X⁶).

In some embodiments, the peptide comprises the Formula Z-X¹-X²-X³-X⁴-X⁵-X⁶-BA, and the peptide comprises one or more of i) Ac-Gly-Asp-Val-Gln-Ala-Glu-BA (SEQ ID NO: 1); ii) Ac-Pro-Asp-Phe-Lys-Pro-Glu-BA (SEQ ID NO: 2); iii) Ac-Gly-Asp-Val-Gln-Ala-Nva-BA (SEQ ID NO: 3); iv) Ac-Gly-Glu-Val-Gln-Ala-Glu-BA (SEQ ID NO: 4); v) Ac-Gly-Abu-Val-Gln-Ala-Glu-BA (SEQ ID NO: 5); vi) Ac-Gly-Asp-Val-Gln-Abu-Glu-BA (SEQ ID NO: 6); vii) Ac-Gly-Asp-Val-Nva-Ala-Glu-BA (SEQ ID NO: 7); viii) Ac-Ser-Asp-Val-Gln-Ala-Glu-BA (SEQ ID NO: 8); ix) Ac-Gly-Asp-Cpg-Gln-Ala-Glu-BA (SEQ ID NO: 9); and x) a salt or solvate thereof; wherein Z is an acetyl group (Ac) covalently bonded to an N-terminus nitrogen on X¹ and the BA is covalently bonded to a carbon atom of X⁶ and/or the BA moiety replaces the —(COOH) of the X⁶. (Cpg) refers to cyclopropylglycine.

In some embodiments, the peptide wherein the peptide comprises Z-X¹-X²-X³-X⁴-X⁵-BA, and the peptide is one or more of: xi) Ac-Asp-Phe-Lys-Pro-Nva-BA (SEQ ID NO: 10); xii) Ac-Asp-Ile-Gln-Ala-Nva-BA (SEQ ID NO: 11); xiii) Ac-Asp-Cpg-Gln-Ala-Nva-BA; and xiv) a salt or solvate thereof, wherein Z is an acetyl group (Ac) covalently bonded to an N-terminus nitrogen on X¹ and the BA is covalently bonded to a carbon atom of X⁵ and/or the BA moiety replaces the —(COOH) of the X⁵. (Cpg) refers to cyclopropylglycine.

In some embodiments, the peptide comprises Z-X¹-X²-X³-X⁴-BA, and the peptide comprises one or more of: xv) thia-Cpg-Gln-Ala-Glu-BA; xvi) Ac-Cpg-Gln-Ala-Glu-BA; xvii) imi-Cpg-Gln-Ala-Glu-BA; xviii) pyr-Cpg-Gln-Ala-Glu-BA; xix) dif-Cpg-Gln-Ala-Glu-BA; xx) dic-Cpg-Gln-Ala-Glu-BA; xxi) Ac-Cpg-Gln-Ala-Nva-BA; xxii) Ac-Cpg-Ala-Pro-Nva-BA; xxiii) Ab-Cpg*-Ala-Pro-Nva-BA; and xxiv) a salt or solvate thereof; wherein Z is an acetyl group (Ac), a thiazole group (thia), an imidazole group (imi), a pyrazine group (pyr), a difluorophenyl group (dif), or a dichlorophenyl group (dic), and Z, when present, is covalently bonded to an N-terminus nitrogen on X¹ and the BA is covalently bonded to a carbon atom of X⁴ and/or the BA moiety replaces the —(COOH) of the X⁴. (Cpg) refers to cyclopropylglycine and (Cpg*) refers to cyclopentylglycine.

In some embodiments, the peptide comprises Z-X¹-X²-X³-BA, and the peptide comprises one or more of: xxv) Ac-Gln-Ala-Nva-BA; and xxvi) a salt or solvate thereof; wherein Z is an acetyl group (Ac) covalently bonded to an N-terminus nitrogen on X¹ and the BA is covalently bonded to a carbon atom of X³ and/or the BA moiety replaces the —(COOH) of the X³.

In some embodiments, the peptide comprises Z-X¹-X²-BA, and the peptide comprises one or more of: xxvii) Ac-Pro-Nva-BA; and xxviii) a salt or solvate thereof; wherein Z is an acetyl group (Ac) covalently bonded to an N-terminus nitrogen on X¹ and the BA is covalently bonded to a carbon atom of X² and/or the BA moiety replaces the —(COOH) of the X².

In some embodiments, a peptide of Formula I is an inhibitor of a CylA serine protease of Enterococcus faecalis, wherein the inhibitor is one of compounds 1-9 and 11-24:

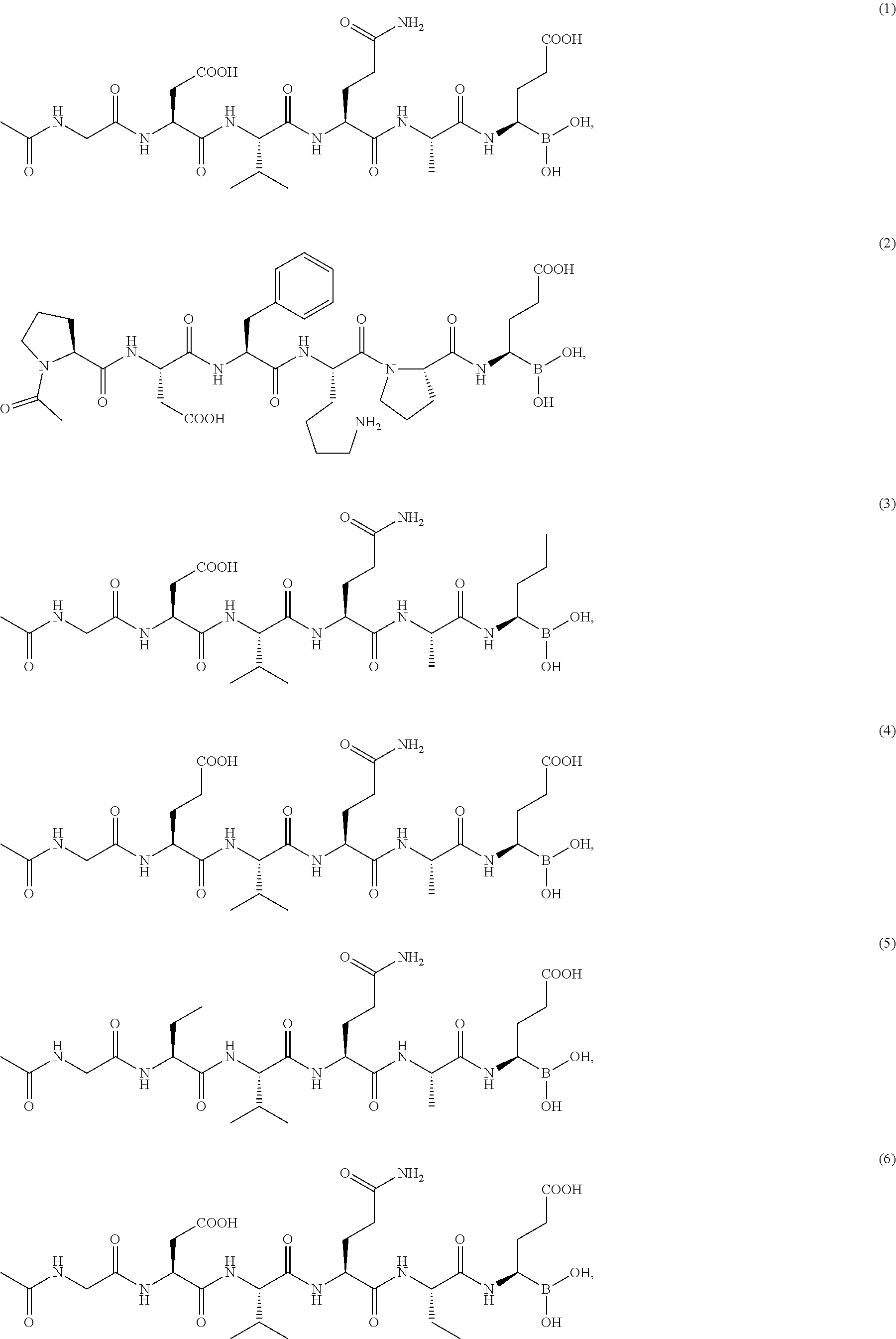

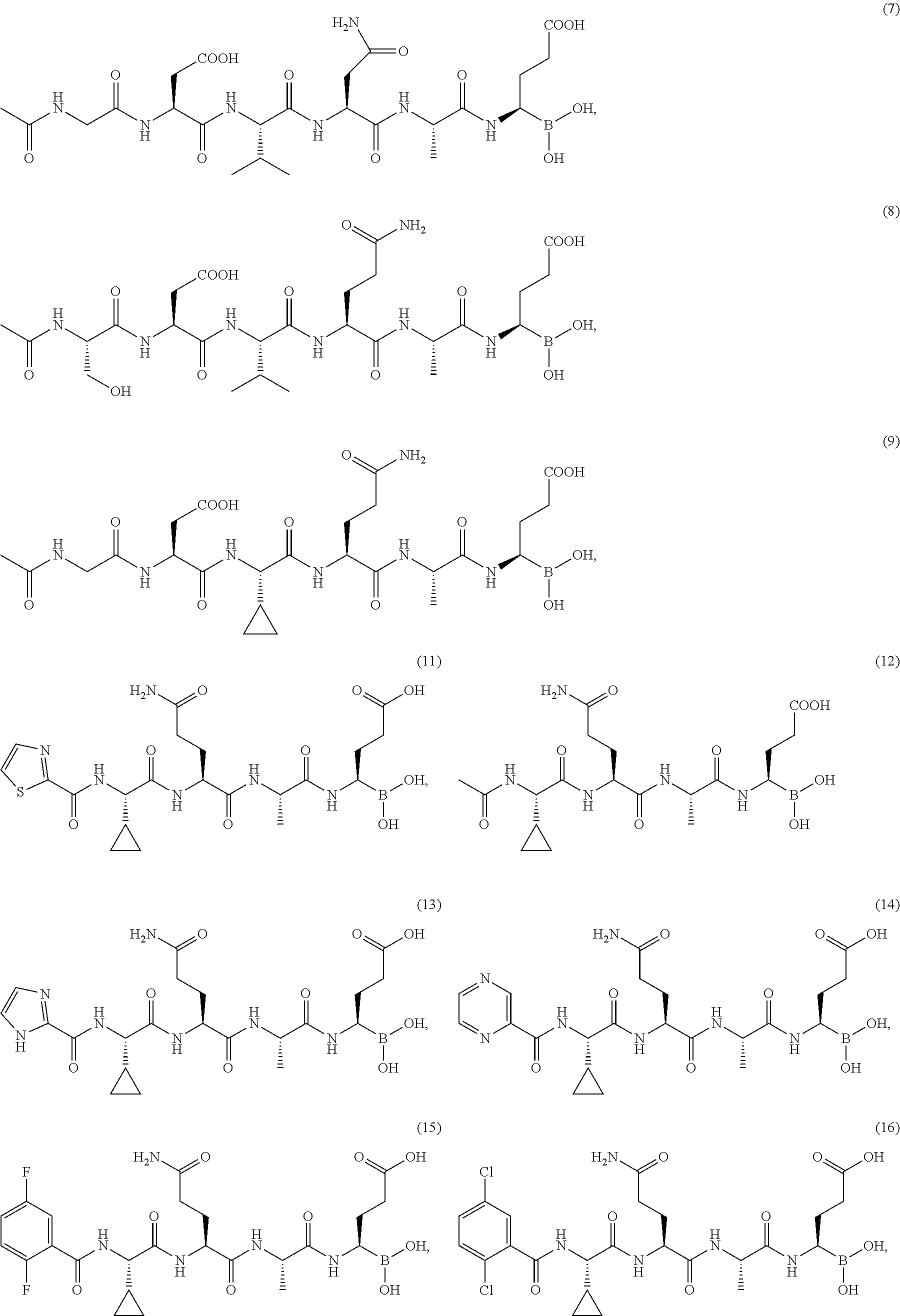

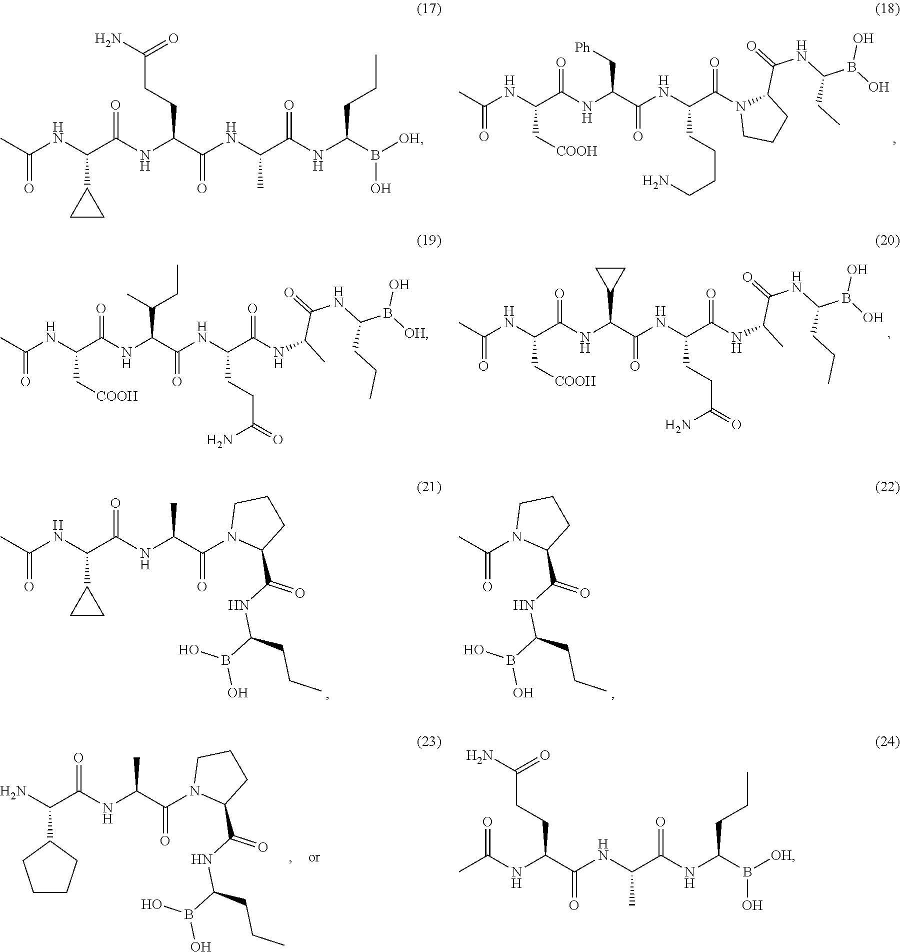

or a salt thereof.

In some embodiments, a method of treating an Enterococcus sp. infection comprising administering to a subject in need thereof a therapeutically effective amount of a peptide or a composition thereof described herein, wherein the peptide eliminates or reduces the release of a cytolysin toxin from the Enterococci sp., thereby treating the infection.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. Inhibitor 1 blocks the His₆-CylA-27-412 catalyzed cleavage of mCylL_{L} and mCylL_{S} preventing the formation of CylL_{L}″ and CylL_{S}″, respectively. A) Peak highlighted in green is the LP+6 amino acids and peak highlighted in orange is CylL_{L}″. B) Peak highlighted in blue is the LP+6 amino acids and peak highlighted in violet is CylL_{S}″. The mCylL_{L} or mCylL_{S} concentration was 10 μM, the His₆-CylA-27-412 concentration was 4 nM, and the concentration of 1 was 1 μM. Reactions were conducted in phosphate buffered saline (PBS, pH=7.4) at room temperature. His₆-CylA-27-412 was incubated with inhibitor for 10 min at room temperature before adding substrate. Reactions were allowed to stand for 30 min before quenching through adjustment to 1% formic acid. Reactions were analyzed directly by LCMS. Extracted ion chromatograms (EIC) for each species were merged into a single trace.

FIG. 2. In vitro pore formation assay for determining inhibitor effectiveness with high sensitivity. The assay uses pore formation as an indicator of toxin maturation. His₆-CylA-27-412 cleaves mCylL_{S} and mCylL_{L} producing CylL_{S}″ and CylL_{L}″, which form pores in pyranine-encapsulated DOPC liposomes at nanomolar concentrations. Pore formation dissipates a pH gradient between the interior of the liposome and the surrounding buffer increasing pyranine fluorescence. Plots of the extent of pore formation as a function of inhibitor concentration gave dose-response curves that were fit using Origin 7 yielding EC₅₀ values.

FIG. 3A-E. FRET assay used to continuously monitor His₆-CylA-27-412 activity and aSEC data describing the process of inhibitor-induced pro-domain release. A) Structure of the FRET substrate and its cleavage by CylA. Samples of FRET substrate (44 μM) in PBS (pH=7.4) before and after treatment with His₆-CylA-27-412 (200 nM) overnight at 37° C. are shown. B) Initial rates of fluorescent peptide formation as a function of initial FRET substrate concentration (2 μM to 125 μM). Non-linear concatenate fitting to the Hill form of the Michaelis Menten model using Origin 7 gave Km, h and the associated errors. C) The relationship between relative enzyme activity (vᵢ/vₒ) and concentration of 2, 4 or 9. Relative enzyme activity was determined by monitoring the rate of cleavage of the FRET peptide in the presence of inhibitor (vi) and comparing the rate to an uninhibited control (vₒ). Non-linear concatenate fitting of data points vᵢ/vₒ to a three-parameter function was used to determine IC₅₀. The colored line represents the calculated fit function. For both B) and C) the experiments were performed in triplicate (n=3) at each concentration and the error bars represent the standard of deviation. D) aSEC analysis of SEC-purified His₆-CylA-27-412 treated with different concentrations of inhibitor 9. E) aSEC traces of SEC-purified His₆-CylA-27-412 before (black) and after (red) incubation with purified samples of pro-domain.

FIG. 4A-C. Structural comparison between the pro domain of CylA (A), the NisB-RRE bound to its substrate (B), and the NHLP-like LP found in the proteusins (C). Structural similarities highlight a convergent strategy for delivering a substrate to its cognate catalytic domain.

FIG. 5A-D. Inhibitor 9 inhibits the hemolytic activity of C+ E. faecalis by decreasing the amount of extractable CylL_{S}″ and reducing the level of cytolysin expression. A) Treatment of an inoculum (OD₆₀₀=0.35) of washed E. faecalis with 100 μM inhibitor 9 (+9), in the presence of washed rabbit erythrocytes, decreased the hemolytic activity relative to an untreated control (−9). Triton X-100 was used as positive control (+T X-100) and untreated cells as negative control (−T X-100). In total, four replicates (n=4) for each condition were performed. B) Assessment of the ability of inhibitor 9 to block the hemolytic activity of live E. faecalis over a range of inhibitor concentrations under conditions described for A). EC₅₀ was determined by fitting the data from at least two replicates with the dose-response function using Origin 7. Error bars represent the standard of deviation. Errors in the EC₅₀ are the errors calculated during the non-linear concatenate fit. C) LCMS analysis of the concentration of CylL_{S}″ extracted from C+ E. faecalis. Overnight cultures of E. faecalis (OD₆₀₀=2.0) were diluted 1000-fold in growth media, treated with 1, 10 or 100 μM of inhibitor 9 and incubated at 37° C. for 20 h. CylL_{S}″ extract concentrations were determined by comparison to an internal standard of ¹⁵N,¹³C-labelled CylL_{S}″. Two technical replicates were recorded for each biological replicate (n=4). Errors bars represent the standard of deviation. D) RT-qPCR data showing the amounts of cylS, cylL, and cylA transcripts extracted from overnight cultures of C+ E. faecalis treated with 100 μM of compound 9 relative to an untreated control (relative gene expression of 1.0). Two technical replicates were collected for each of the two biological replicates (n=2). Error bars represent the standard of deviation.

### DETAILED DESCRIPTION OF THE INVENTION

The enterococcal cytolysin is a two-component lanthipeptide secreted by Enterococcus faecalis that functions as a virulence factor. The lanthipeptide is responsible for hemolytic phenotypes of E. faecalis and its role in disease has been known for almost a century. The natural producer of cytolysin is a commensal organism and a member of the human gut microbiome and, although its prevalence among humans has not been firmly established, it is thought that a significant fraction of the human population hosts C+ E. faecalis. In healthy hosts, C+ E. faecalis are not known to pose a health risk. In unhealthy hosts, such as those diagnosed with alcoholic hepatitis, the presence of C+ E. faecalis is implicated in negative clinical outcomes, resulting in 89% of C+ patients with alcoholic hepatitis dying within 180 days of admission, compared to 3.8% of cytolysin negative patients (C−). In the same study, it was shown that targeted destruction of the C+ E. faecalis using phages attenuated liver toxicity in mouse models. These results suggest that targeted inhibition of the cytolysin biosynthetic pathway could help in treating this deadly disease state.

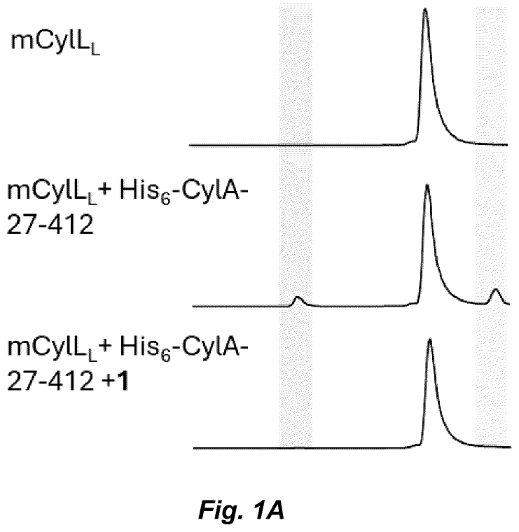

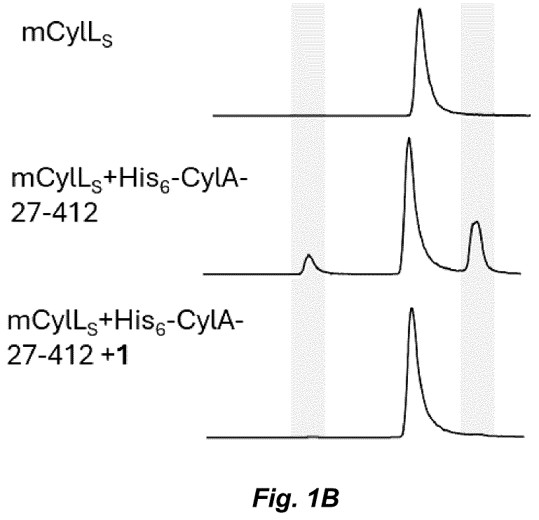

The biosynthesis of cytolysin has been studied in detail and the process is summarized in Moreira et al, FIG. 1. The cytolysin BGC constituted the first reported examples of class II lanthipeptides. As with most RiPPs, the gene-encoded substrate is significantly longer than the mature natural product and proteases are involved in the maturation process. The full-length substrate is comprised of two sections: the leader peptide (LP), which is involved in substrate recognition by the biosynthetic enzymes and modulating substrate toxicity, and the core peptide, which is the section of the peptide that undergoes post-translational modification and comprises the mature natural product. The enterococcal cytolysin BGC contains two precursor sequences that encode for CylL_{L} and CylL_{S} that are post-translationally modified by a single class II lanthipeptide synthetase called CylM. This enzyme dehydrates Thr and Ser residues yielding dehydrobutyrine (Dhb) and dehydroalanine (Dha), respectively, which act as acceptors for enzyme-guided Michael-type additions by Cys side chains forming LL-methyllanthionine (MeLan) and LL- and DL-lanthionine (Lan) bridges. The presence and stereochemistry of these lanthionine residues are important for toxin activity. The full-length modified precursors, mCylL_{L} and mCylL_{S}, are exported and the main part of the LP is removed by a bifunctional transporter-protease, CylB, at GlySer sequences yielding CylL_{L}′ and CylL_{S}′. An extracellular protease, CylA, then cleaves these biosynthetic intermediates yielding the mature natural products CylL_{L}″ and CylL_{S}″, which cooperatively kill both bacterial and eukaryotic cells at nanomolar concentrations through an unknown mechanism, but which appears to involve membrane disruption.

Cytolysin is expressed in response to cell density through a unique quorum sensing mechanism made up of two proteins, CylR1 and CylR2. CylR1 is a putative membrane protein that is thought to interact with the transcription factor CylR2 in a manner that influences DNA binding. Upon detection of free CylL_{S}″, CylR1/CylR2 promotes the transcription of the cytolysin BGC. At low cell densities, cytolysin is expressed at a low level and, in the absence of encroaching cell membranes, CylL_{S}″ and CylL_{L}″ cooperatively aggregate preventing CylL_{S}″ from stimulating CylR1/R2. When the cell density rises, CylL_{L}″ is thought to insert into the surrounding cell membranes leaving CylL_{S}″ to accumulate in solution. The accumulated CylL_{S}″ then interacts with CylR1 in a way that changes the DNA binding of CylR2, thus stimulating cytolysin expression.

Based on the current knowledge of cytolysin biosynthesis, several enzymes involved in the maturation of cytolysin could be targeted to control cytolysin virulence. Previous studies demonstrated that mCylL_{S}, mCylL_{L}, CylL_{S}′, and CylL_{L}′ are not hemolytically active and do not stimulate cytolysin expression. Therefore, CylM, CylB, CylA and CylR1/R2 are potential targets to prevent production of active toxin; however, CylA is a particularly attractive target due to its extracellular localization and its similarity to other proteases that have been successfully targeted by anti-virulence strategies.

CylA is a subtilisin-like serine protease that is essential for toxin activation. Like many proteases, CylA is translated as a proenzyme that undergoes activation through self-cleavage. Once cleaved, the C-terminal catalytic domain of CylA is thought to non-covalently bind to its pro-domain based on co-purification of these two domains and similarity to a related protease, LicP, which was crystallized with the pro-domain non-covalently bound to the active site. The active form of CylA then recognizes the GDVQAE (SEQ ID NO: 13) sequence, which it removes from CylL_{L}′ and CylL_{S}′ to produce the active toxin. In RiPP biosynthesis, the C-terminal amino acid in the LP is numbered-1 and other residues in the LP are numbered backwards from this position with negative numbers. The putative self-cleavage recognition sequence (PDFKPE) (SEQ ID NO: 12) and the substrate recognition sequence (GDVQAE) (SEQ ID NO: 13) are different but do share Glu and Asp at positions −1 and −5, respectively. Previously, it was suggested that Glu-1 is necessary for positioning the substrate in the enzyme active site through an interaction with the side chain of His₂₇₅-a His that flanks the active site. Although in vivo LP removal is a two-step process involving first CylB and then CylA, in vitro CylA can remove the entire LP in one step.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991), and Hawley's Condensed Chemical Dictionary 14th Edition, by R. J. Lewis, John Wiley & Sons, New York, N.Y., 2001. General laboratory techniques (DNA extraction, RNA extraction, cloning, cell culturing, etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J M B, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, ‘patient’ may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013. Also see U.S. Pat. No. 12,264,172 to Metcalf et al.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term amino acids described herein are among the 20 naturally occurring amino acids that have the L-configuration unless indicated otherwise. An amino acid that has the D-configuration is denoted symbolically with a superscript capital “D” preceding its name or its standard abbreviation. For example, the D-configuration of alanine with be abbreviated as ^{D}alanine, ^{D}Ala, or ^{D}A; and it may also be indicated as D-alanine or a lower case single letter abbreviation, e.g., “a” for D-alanine.

As used herein, the term “non-polar and uncharged amino acid” refers to alanine, valine, leucine, isoleucine, methionine, phenylalanine, tryptophan, proline, cyclopropylglycine, 2-aminobutyric acid, and norvaline. The term “polar and uncharged amino acid” refers to serine, threonine, cytosine, tyrosine, asparagine, glutamine, and glycine.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “IC₅₀” is generally defined as the concentration required to inhibit a specific biological or biochemical function by half, or to kill 50% of the cells in a designated time period, typically 24 hours.

Aspects of the invention are described in Moreira et al., Nat Commun 16, 6936 (2025), doi.org/10.1038/s41467-025-62161-7, including any supplemental information published therewith, which publication is incorporated herein by reference in its entirety.

### Embodiments of the Invention

The enterococcal cytolysin is a toxic, two-component ribosomally synthesized and post-translationally modified peptide (RiPP) that is produced by Enterococcus faecalis. Cytolysin-producing (C+) E. faecalis typically resides in the gut microbiome in a commensal role, but recent studies have correlated the presence of cytolysin produced by gut-dwelling C+ E. faecalis with negative clinical outcomes in patients diagnosed with alcoholic hepatitis. To potentially combat cytolysin virulence, we applied knowledge of cytolysin biosynthesis to develop small molecule inhibitors of its maturation. CylA, an extracellular serine protease that is essential for toxin activation, was chosen as a target. Using the substrate and self-cleavage sequences of CylA as scaffolds, a series of α-aminopeptide boronic acids was designed and synthesized. In vitro studies demonstrated that these inhibitors blocked cytolysin maturation at low micromolar to low nanomolar concentrations. A crystal structure of CylA provided unexpected structural insights into substrate recognition, autocatalytic activation of the enzyme, and toxin maturation. Experiments with C+ E. faecalis demonstrated that the designed inhibitor can block hemolytic activity, reduce the amount of extractable cytolysin, and attenuate expression of the cytolysin biosynthetic gene cluster without impeding cell growth. These studies validate CylA as an accessible anti-virulence target and provide a potential route to the development of treatments for cytolysin-induced disease states.

Accordingly, the disclosure provides for certain peptides that are modified at the C-terminal end via a boronic acid moiety (—B(OH)₂) covalently bonded to a carbon atom of the C-terminal amino acid or replaces the —(COOH) (i.e., the carboxylic acid) of the C-terminal amino acid, and optionally, also may be modified at the N-terminal end via an acyl moiety, a thiazole moiety, an imidazole moiety, a pyrazine moiety, a difluorophenyl moiety, or a dichlorophenyl moiety covalently bonded to covalently bonded to an N-terminus nitrogen atom of the N-terminal amino acid.

In some embodiments, a peptide comprises, from an N-terminus to a C-terminus, the Formula I:

Z-X¹-X²-X³-X⁴-X⁵-X⁶-BA (I),

wherein Z is an acetyl group, a thiazole group, an imidazole group, a pyrazine group, a difluorophenyl group, a dichlorophenyl group, or absent, wherein Z, when present, is covalently bonded to an N-terminus nitrogen on X¹; X¹ is Gly, Pro, Ser, cyclopropylglycine (Cpg), cyclopentylglycine (Cpg*), Asp, Gln, or Ala; X² is Asp, Glu, Gln, Ala, Phe, Ile, Cpg, Norvaline (Nva), or 2-Aminobutyric acid (Abu); X³ is Lys, Gln, Val, Phe, Cpg, Ala, Pro, Nva, or absent; X⁴ is Glu, Gln, Lys, Pro, Ala, Nva, or absent; X⁵ is Ala, Pro, Nva, Abu, or absent; X⁶ is Gly, Glu or Nva; and BA is a boronic acid moiety (—B(OH)₂) covalently bonded to a carbon atom of an amino acid at the C-terminus of the peptide and/or the BA moiety replaces the —(COOH) of the amino acid at the C-terminus of the peptide.

In some embodiments, the peptide comprises Z-X¹-X²-X³-X⁴-X⁵-X⁶-BA, and the peptide comprises one or more of i) Ac-Gly-Asp-Val-Gln-Ala-Glu-BA (SEQ ID NO: 1); ii) Ac-Pro-Asp-Phe-Lys-Pro-Glu-BA (SEQ ID NO: 2); iii) Ac-Gly-Asp-Val-Gln-Ala-Nva-BA (SEQ ID NO: 3); iv) Ac-Gly-Glu-Val-Gln-Ala-Glu-BA (SEQ ID NO: 4); v) Ac-Gly-Abu-Val-Gln-Ala-Glu-BA (SEQ ID NO: 5); vi) Ac-Gly-Asp-Val-Gln-Abu-Glu-BA (SEQ ID NO: 6); vii) Ac-Gly-Asp-Val-Nva-Ala-Glu-BA (SEQ ID NO: 7); viii) Ac-Ser-Asp-Val-Gln-Ala-Glu-BA (SEQ ID NO: 8); ix) Ac-Gly-Asp-Cpg-Gln-Ala-Glu-BA (SEQ ID NO: 9); and x) a salt or solvate thereof; wherein Z is an acetyl group (Ac) covalently bonded to an N-terminus nitrogen on X¹ and the BA is covalently bonded to a carbon atom of X⁶ and/or the BA moiety replaces the —(COOH) of the X⁶. (Cpg) refers to cyclopropylglycine.

In some embodiments, the peptide wherein the peptide comprises Z-X¹-X²-X³-X⁴-X⁵-BA, and the peptide is one or more of: xi) Ac-Asp-Phe-Lys-Pro-Nva-BA (SEQ ID NO: 10); xii) Ac-Asp-Ile-Gln-Ala-Nva-BA (SEQ ID NO: 11); xiii) Ac-Asp-Cpg-Gln-Ala-Nva-BA; and xiv) a salt or solvate thereof, wherein Z is an acetyl group (Ac) covalently bonded to an N-terminus nitrogen on X¹ and the BA is covalently bonded to a carbon atom of X⁵ and/or the BA moiety replaces the —(COOH) of the X⁵. (Cpg) refers to cyclopropylglycine.

In some embodiments, the peptide comprises Z-X¹-X²-X³-X⁴-BA, and the peptide comprises one or more of: xv) thia-Cpg-Gln-Ala-Glu-BA; xvi) Ac-Cpg-Gln-Ala-Glu-BA; xvii) imi-Cpg-Gln-Ala-Glu-BA; xviii) pyr-Cpg-Gln-Ala-Glu-BA; xix) dif-Cpg-Gln-Ala-Glu-BA; xx) dic-Cpg-Gln-Ala-Glu-BA; xxi) Ac-Cpg-Gln-Ala-Nva-BA; xxii) Ac-Cpg-Ala-Pro-Nva-BA; xxiii) Ab-Cpg*-Ala-Pro-Nva-BA; and xxiv) a salt or solvate thereof; wherein Z is an acetyl group (Ac), a thiazole group (thia), an imidazole group (imi), a pyrazine group (pyr), a difluorophenyl group (dif), or a dichlorophenyl group (dic), and Z, when present, is covalently bonded to an N-terminus nitrogen on X¹ and the BA is covalently bonded to a carbon atom of X⁴ and/or the BA moiety replaces the —(COOH) of the X⁴. (Cpg) refers to cyclopropylglycine and (Cpg*) refers to cyclopentylglycine.

In some embodiments, the peptide comprises Z-X¹-X²-X³-BA, and the peptide comprises one or more of: XXV) Ac-Gln-Ala-Nva-BA; and xxvi) a salt or solvate thereof, wherein Z is an acetyl group (Ac) covalently bonded to an N-terminus nitrogen on X¹ and the BA is covalently bonded to a carbon atom of X³ and/or the BA moiety replaces the —(COOH) of the X³.

In some embodiments, the peptide comprises Z-X¹-X²-BA, and the peptide comprises one or more of: xxvii) Ac-Pro-Nva-BA; and xxviii) a salt or solvate thereof, wherein Z is an acetyl group (Ac) covalently bonded to an N-terminus nitrogen on X¹ and the BA is covalently bonded to a carbon atom of X² and/or the BA moiety replaces the —(COOH) of the X².

In some embodiments, the peptide comprises one or more of a) Ac-Gly-Asp-Val-Gln-Ala-Glu-BA (SEQ ID NO: 1); b) Ac-Pro-Asp-Phe-Lys-Pro-Glu-BA (SEQ ID NO: 2); c) Ac-Gly-Asp-Val-Gln-Ala-Nva-BA (SEQ ID NO: 3); d) Ac-Gly-Glu-Val-Gln-Ala-Glu-BA (SEQ ID NO: 4); e) Ac-Gly-Abu-Val-Gln-Ala-Glu-BA (SEQ ID NO: 5); f) Ac-Gly-Asp-Val-Gln-Abu-Glu-BA (SEQ ID NO: 6); g) Ac-Gly-Asp-Val-Nva-Ala-Glu-BA (SEQ ID NO: 7); h) Ac-Ser-Asp-Val-Gln-Ala-Glu-BA (SEQ ID NO: 8); i) Ac-Gly-Asp-Cpg-Gln-Ala-Glu-BA (SEQ ID NO: 9); j) Ac-Asp-Phe-Lys-Pro-Nva-BA (SEQ ID NO: 10); k) Ac-Asp-Ile-Gln-Ala-Nva-BA (SEQ ID NO: 11); 1) Ac-Asp-Cpg-Gln-Ala-Nva-BA; m) thia-Cpg-Gln-Ala-Glu-BA; n) Ac-Cpg-Gln-Ala-Glu-BA; o) imi-Cpg-Gln-Ala-Glu-BA; p) pyr-Cpg-Gln-Ala-Glu-BA; q) dif-Cpg-Gln-Ala-Glu-BA; r) dic-Cpg-Gln-Ala-Glu-BA; s) Ac-Cpg-Gln-Ala-Nva-BA; t) Ac-Cpg-Ala-Pro-Nva-BA; u) Ab-Cpg*-Ala-Pro-Nva-BA; v) Ac-Gln-Ala-Nva-BA; w) Ac-Pro-Nva-BA; and salt or solvate thereof, wherein (Ac) is an acetyl group, (thia) is a thiazole group, (imi) is an imidazole group, (pyr) is a pyrazine group, (dif) is a difluorophenyl group, (dic) is a dichlorophenyl group, the Ac, the thia, the imi, the pyr, the dif, and the dic are covalently bonded to an N-terminus nitrogen of the N-terminal amino acid and the BA is covalently bonded to a carbon atom of the C-terminal amino acid and/or the BA moiety replaces the —(COOH) of the C-terminal amino acid.

In some embodiments, the peptide may be the free peptide or salt or solvate thereof. In some embodiments the peptide is included in a composition comprising the peptide or a salt or solvate thereof and a pharmaceutically acceptable carrier, excipient, or diluent.

The disclosure also provides for methods of treating an Enterococcus sp. infection comprising administering to a subject in need thereof a therapeutically effective amount of a peptide or a salt or solvate thereof or a composition comprising the peptide or a salt or solvate thereof, wherein the peptide stops or reduces the release of a cytolysin toxin from the Enterococci sp., thereby treating the infection.

In some embodiments, the Enterococcus sp., comprises one or more of E. faecalis, E. faecium, E. avium, E. caccae, E. casseliflavus, E. dispar, E. durans, E. gallinarum, E. hirae, and E. raffinosus. In some embodiments, the Enterococcus sp., is E. faecalis and/or E. faecium.

Embodiments of the disclosure also provide an inhibitor of a CylA serine protease of Enterococcus faecalis, wherein the inhibitor is one of compounds 1-9:

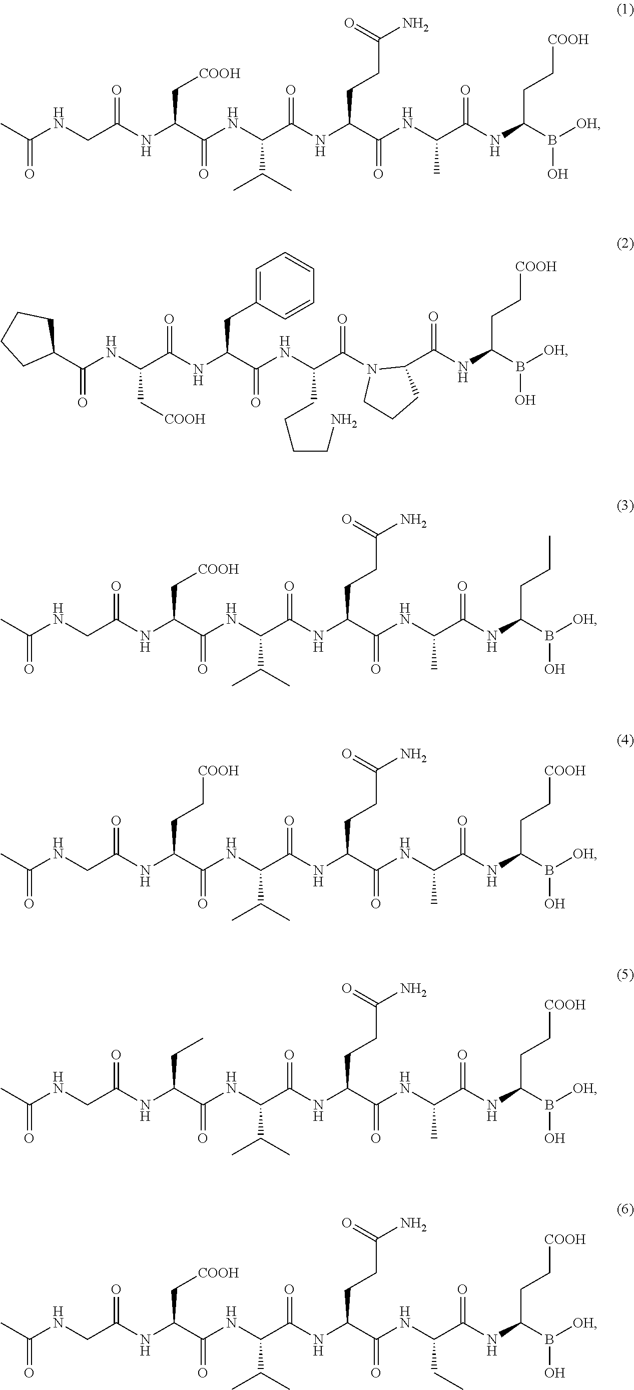

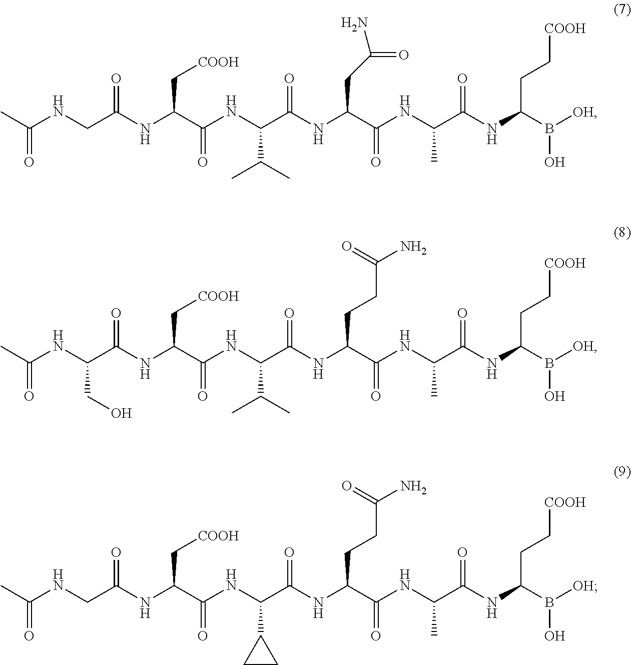 [SMILES: CC(=O)NCC(=O)N[C@@H](CC(=O)O)C(=O)N[C@H](C(=O)N[C@@H](CC(N)=O)C(=O)N[C@@H](C)C(=O)N[C@@H](CCC(=O)O)B(O)O)C(C)C ; CC(=O)N[C@@H](CO)C(=O)N[C@@H](CC(=O)O)C(=O)N[C@H](C(=O)N[C@@H](CCC(N)=O)C(=O)N[C@@H](C)C(=O)N[C@@H](CCC(=O)O)B(O)O)C(C)C ; CC(=O)NCC(=O)N[C@@H](CC(=O)O)C(=O)N[C@H](C(=O)N[C@@H](CCC(N)=O)C(=O)N[C@@H](C)C(=O)N[C@@H](CCC(=O)O)B(O)O)C1CC1]

or a salt or solvate thereof.

In some embodiments, two or more inhibitors selected from compounds 1-9 can be used in a method for inhibiting a CylA serine protease.

In some embodiments, the inhibitor of a CylA serine protease of Enterococcus faecalis is one of compounds 11-24:

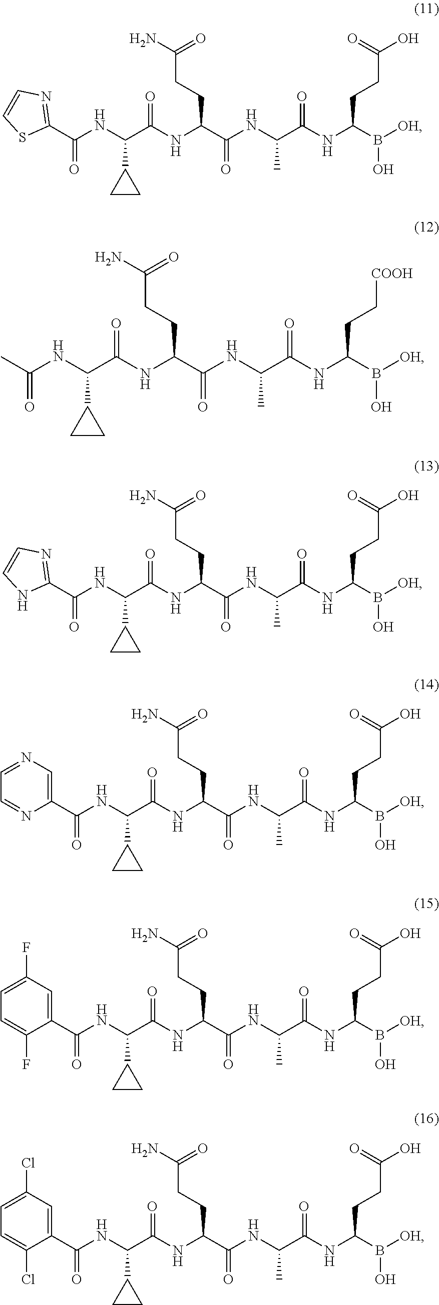

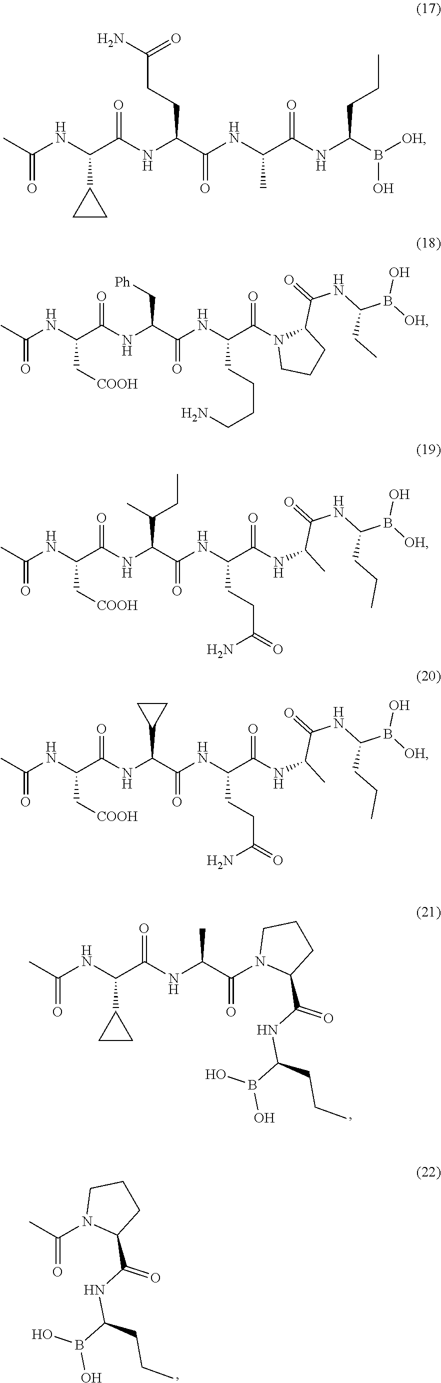

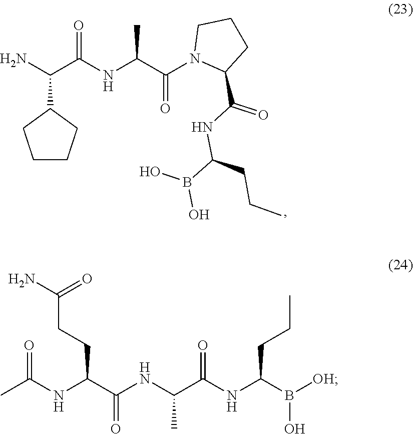

or a salt or solvate thereof. In some embodiments, two or more inhibitors selected from compounds 11-24 can be used in a method for inhibiting a CylA serine protease.

In some embodiments, a composition for inhibiting a CylA serine protease may comprise an inhibitor of any one of structures 1-9 and 11-24, or any one of 1-3 and 6-9, and pharmaceutically acceptable carrier, excipient, or diluent. In some embodiments, a composition may include two or more CylA serine protease inhibitors.

In some embodiments, the peptide and/or the inhibitors of a CylA serine protease of Enterococcus faecalis has an EC₅₀ of about 5 nM to about 150 nM, about 10 nM to about 120 nM, or about 10 nM to about 80 nM.

In some embodiments, the peptide and/or the inhibitors of a CylA serine protease of Enterococcus faecalis has an IC₅₀ of about 0.01 μM to about 40 μM, or about 0.1 μM to about 20 μM.

### Pharmaceutical Formulations

The compounds and compositions described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and β-glycerophosphate. Suitable inorganic salts may also be formed, including hydrochloride, halide, sulfate, nitrate, bicarbonate, and carbonate salts. Also see, for example, U.S. Pat. No. 12,116,386 to Chappell et al.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium, or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical, or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose, or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the disclosure provides a composition comprising a compound of the disclosure formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day.

The sub-dose itself may be further divided, e.g., into a number of discrete loosely spaced administrations; such as multiple inhalations from an insufflator or by application of a plurality of drops into the eye.

### Synthesis of Peptide Boronic Acid Inhibitors for CylA.

Boronic acids are attractive war heads for inhibitors of serine proteases and several FDA approved drugs contain boronic acids. CylA is a subtilisin-like serine protease possessing a Ser-Asp-His catalytic triad and proteases of this type have been targeted by boronic acid containing peptides leading to beneficial decreases in enzyme activity in vivo. CylA cleaves after the N-terminal hexapeptide sequence GDVQAE (SEQ ID NO: 13), and probably recognizes the PDFKPE (SEQ ID NO: 12) sequence at its self-cleavage site. We envisioned replacing the carboxylic acid terminus of each hexapeptide sequence with a boronic acid to generate inhibitors that bind covalently but reversibly to CylA preventing conversion of CylL_{S}′ and CylL_{L}′ to their mature counterparts.

Boronic acid-containing hexapeptide inhibitors were synthesized through a convergent approach detailed in Scheme 1.

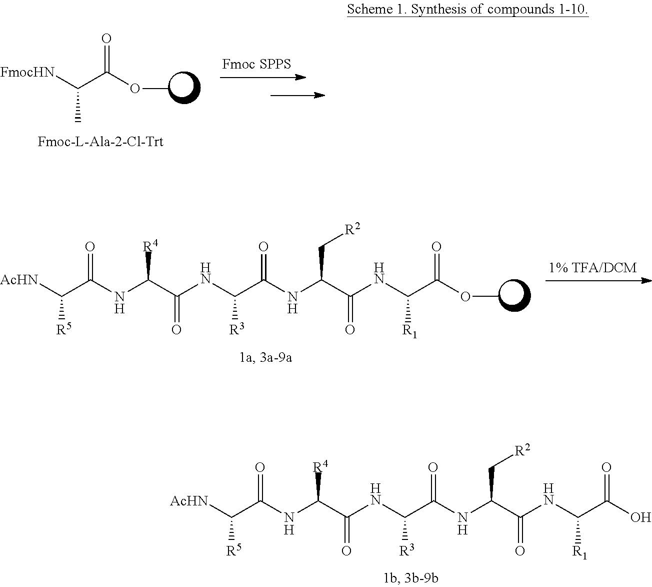

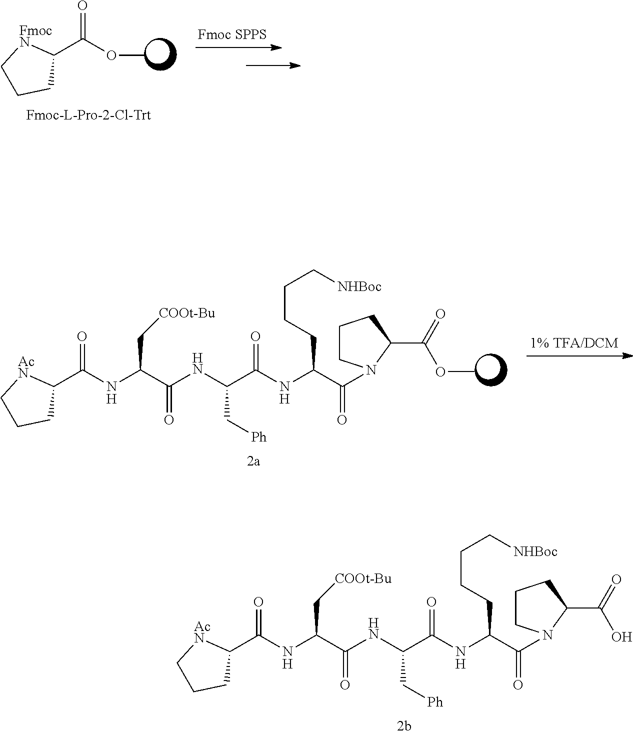

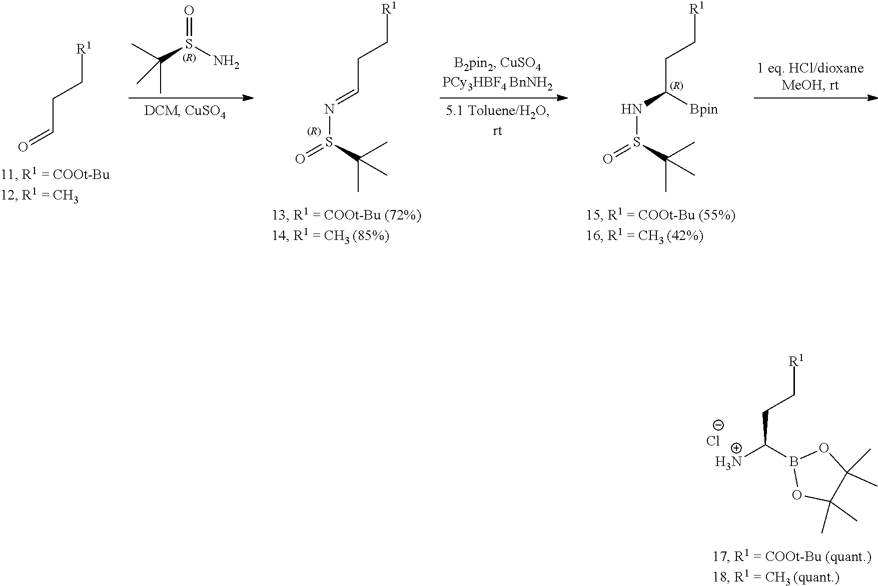

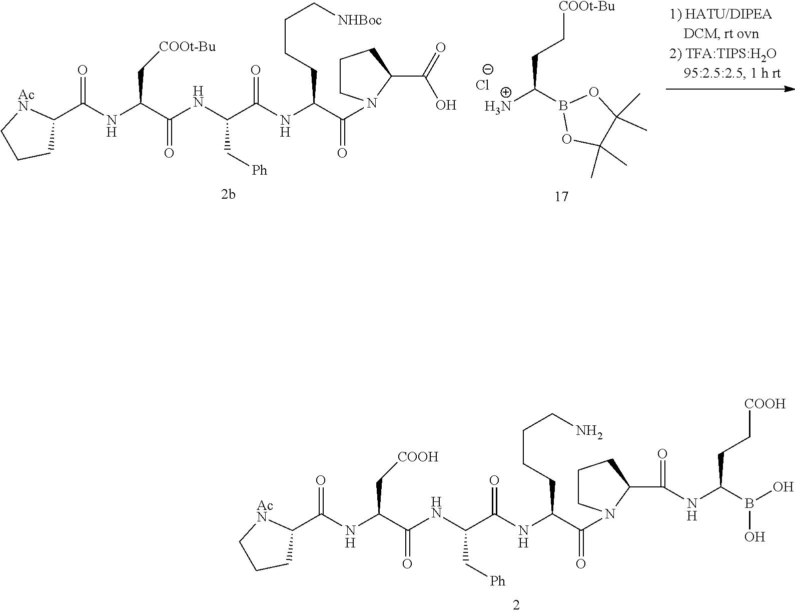

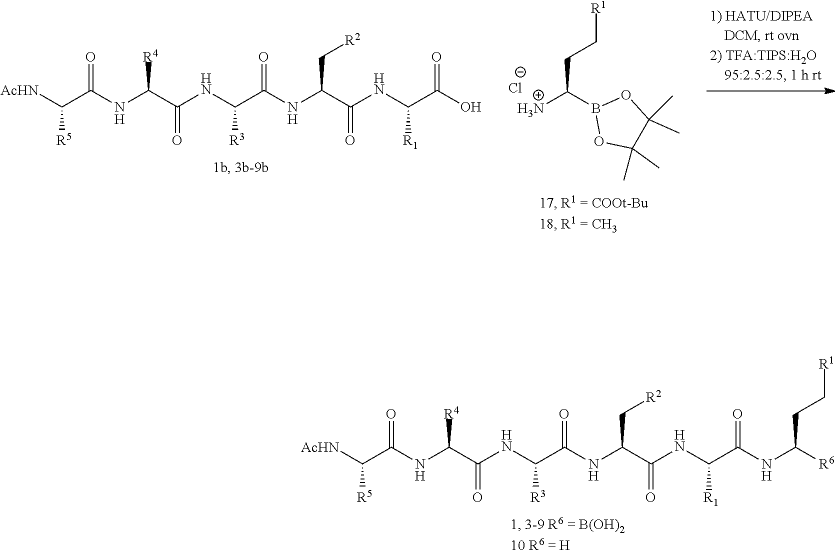

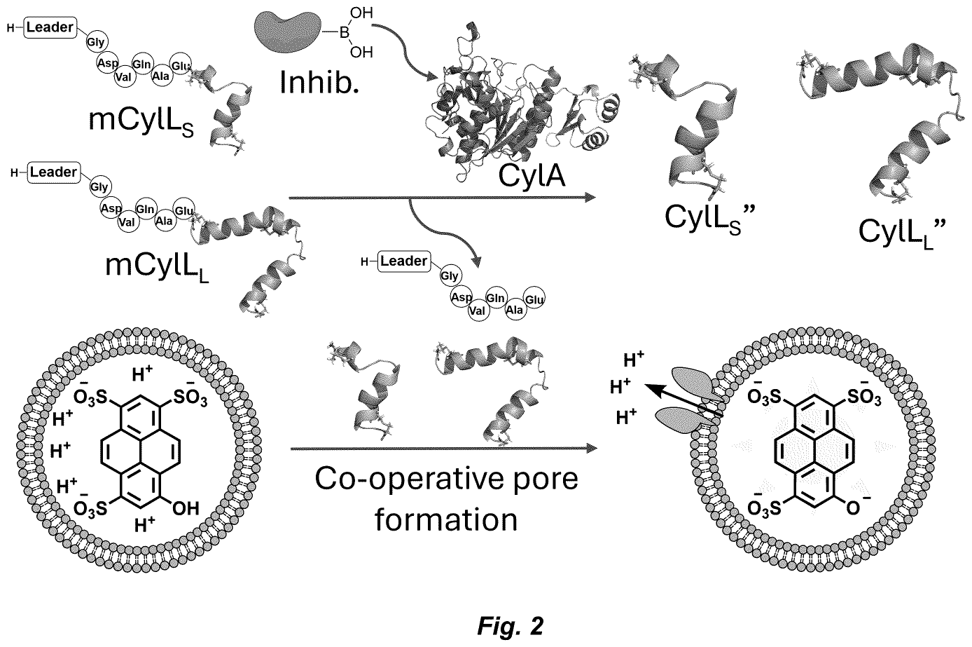

The pentapeptide fragments were prepared using Fmoc solid phase peptide synthesis (SPPS) starting from 2-chlorotrityl(2ClTrt) resin loaded with Fmoc-L-Ala or Fmoc-L-Pro. Standard Fmoc SPPS conditions were used to extend to the −6 position, which was acetylated under standard conditions yielding peptides 1a-9a. Each peptide was removed from the solid support with 1% trifluoroacetic acid (TFA) in dichloromethane (DCM) keeping all the side chain protecting groups intact yielding protected peptides 1b-9b. The side chains of 1a-9a and 1b-9b match the compound with the same number presented in Table 1; for example, la was the precursor to 1b and 1b was the precursor to 1. α-Amino boronic esters were prepared enantioselectivity starting from aldehydes 11 and 12, which were converted to chiral N-tert-butanesulfinyl imines 13 and 14, respectively, through treatment with CuSO₄/DCM in the presence of (R)-(+)-2-methyl-2-propanesulfinamide. Compounds 13 and 14 were subjected to diastereoselective borylation yielding a-sulfinamide boronic esters 15 and 16, respectively. Removal of the sulfinamide from 15 and 16 occurred readily in the presence of HCl/dioxane/MeOH yielding 17 and 18, respectively, which were immediately coupled to 1b-9b using 1-[bis(dimethylamino)methylene]-1H-1,2,3-triazolo[4,5-b]pyridinium 3-oxid hexafluorophosphate (HATU) and N,N-diisopropylethylamine (DIPEA) in DCM. Following aqueous workup, the crude protected peptides were globally deprotected by treatment with TFA/H₂O/triisopropylsilane (TIPS), then precipitated from cold diethyl ether and purified by ultra-high-performance liquid chromatography (UHPLC) yielding 1-9 (See Moreira et al., Supplementary FIGS. 1 and 2). During treatment of 9b with TFA/TIPS/H₂O, up to 30% of the deprotected peptide underwent protodeboronation, giving peptide 10, which was successfully isolated and used to assess the impact of the boronic acid warhead in vitro.

### Peptide Boronic Acids are Potent Inhibitors of His₆-CylA-27-412 In Vitro.

We first tested the ability of compound 1, which contains the natural substrate recognition sequence, to inhibit the CylA-catalyzed cleavage of mCylL_{S} and mCylL_{L} in vitro using liquid chromatography with mass spectrometric detection (LCMS) (FIG. 1). We expressed CylA in Escherichia coli with an N-terminal His₆-tag and removed the secretion signal that is present in

WT CylA. After purification by metal affinity chromatography, incubation of each substrate with this purified His₆-CylA-27-412 resulted in partial conversion to the mature core peptide after 30 min at room temperature. Addition of compound 1 (1 μM) completely blocked His₆-CylA-27-412-catalyzed cleavage over the 30 min reaction time.

Cytolysin causes hemolysis and lyses a variety of eukaryotic cells. Additionally, CylA aggregates and is thought to associate primarily with the membrane of the producing cell. To mimic the maturation of cytolysin in vitro, a coupled enzyme assay was designed in which mCylL_{L} and mCylL_{S} were combined in a 1:1 ratio and treated with His₆-CylA-27-412 in the presence of pyranine containing liposomes in buffer of pH 7.4 (FIG. 2). Within the liposomes was a buffer of lower pH (6.0), and therefore any pore formation or membrane disruption would result in an increase in internal pH, which increases the fluorescence signal of the pyranine. In this way, pyranine fluorescence can be correlated to toxin activation allowing for the determination of EC₅₀ values. Using this approach, the inhibitory activities of compounds 1-10 were evaluated (Table 1).

Compound 1, which mimics the natural recognition sequence of the substrate, inhibited liposome lysis with an EC₅₀ of 30±3 nM. Peptide 2, which is based on the self-cleavage sequence of CylA, showed an EC₅₀ of 117±16 nM, about 4-fold higher than 1 suggesting that the GDVQAE (SEQ ID NO: 13) sequence of the substrate can displace the PDFKPE (SEQ ID NO: 12) sequence of inhibitor 2 allowing for weaker inhibition of toxin activation. Comparing the activity of 1 and 3, replacing the C-terminal Glu-mimicking boronate with a norvaline-mimicking boronate did not significantly change the EC₅₀. This observation was unexpected since both the substrate and self-cleavage sequence contain Glu-1, the related enzyme LicP requires Glu at-1 of its substrate, and an important salt bridge between the side chain carboxyl group of Glu-1 and His275 of CylA has been proposed. Replacement of Asp-5 in 1 with a Glu residue (4) resulted in a 25-fold increase in EC₅₀ and replacement of the same Asp with 2-aminobutyric acid (Abu, 5) increased the EC₅₀ 224-fold. These two results suggest that Asp-5 is involved in an important interaction with the enzyme, consistent with the substrate interaction model proposed for LicP, which contains a salt bridge between Asp-5 and a proximal Lys residue. Replacement of Ala-2 in compound 1 with Abu (6) resulted in a ~2-fold increase in EC₅₀. Switching Gln-3 to Asn (7) marginally increased the EC₅₀ and replacement of Gly-6 with Ser (8) produced a similar increase suggesting that some variability is tolerated at these positions. We hypothesized that converting Val to cyclopropylglycine (Cpg) could increase inhibitor effectiveness through conformational constriction of the side chain and indeed compound 9 had an EC₅₀ of 12.5±1.4 nM, which is 2-fold more effective than compound 1 at inhibiting pore formation. The protodeboronated form of 9, compound 10, was found to also inhibit pore formation but it was 115-fold less effective than boronic acid containing 9.

TABLE 1
Structures and EC₅₀ values of inhibitors 1-10. Errors represent the error in the non-
linear concatenate fit of three separate trials (n = 3).

| Compound | Structure | EC50 (nM) |
|---|---|---|
| 1 | 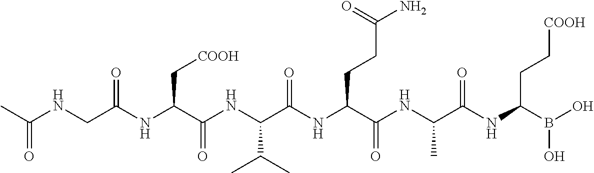 [SMILES: CC(=O)NCC(=O)N[C@@H](CC(=O)O)C(=O)N[C@H](C(=O)N[C@@H](CCC(N)=O)C(=O)N[C@@H](C)C(=O)N[C@@H](CCC(=O)O)B(O)O)C(C)C] | 30 ± 3 |
| 2 | 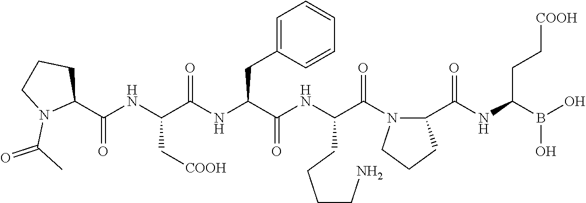 [SMILES: CC(=O)N1CCC[C@H]1C(=O)N[C@@H](CC(=O)O)C(=O)N[C@@H](Cc1ccccc1)C(=O)N[C@@H](CCCCN)C(=O)N1CCC[C@H]1C(=O)N[C@@H](CCC(=O)O)B(O)O] | 117 ± 16 |
| 3 | 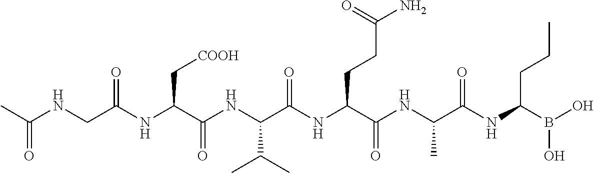 [SMILES: CCC[C@H](NC(=O)[C@H](C)NC(=O)[C@H](CCC(N)=O)NC(=O)[C@@H](NC(=O)[C@H](CC(=O)O)NC(=O)CNC(C)=O)C(C)C)B(O)O] | 35 ± 6 |
| 4 | 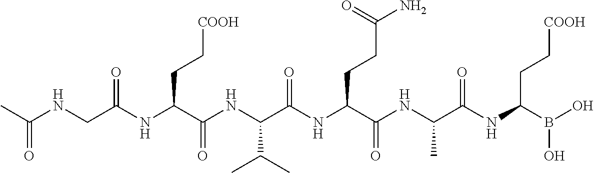 [SMILES: CC(=O)NCC(=O)N[C@@H](CCC(=O)O)C(=O)N[C@H](C(=O)N[C@@H](CCC(N)=O)C(=O)N[C@@H](C)C(=O)N[C@@H](CCC(=O)O)B(O)O)C(C)C] | 748 ± 80 |
| 5 | 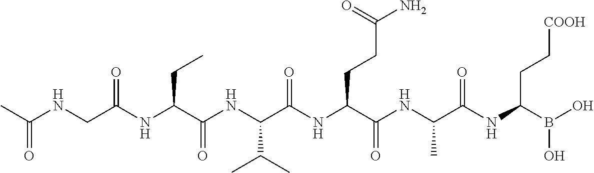 [SMILES: CC[C@H](NC(=O)CNC(C)=O)C(=O)N[C@H](C(=O)N[C@@H](CCC(N)=O)C(=O)N[C@@H](C)C(=O)N[C@@H](CCC(=O)O)B(O)O)C(C)C] | (6.7 ± 0.8) × 10³ |
| 6 | 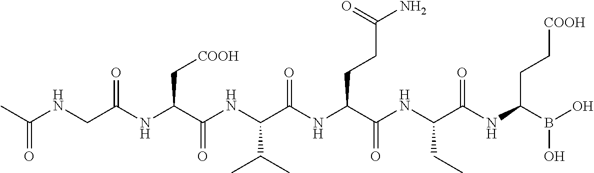 [SMILES: CC[C@H](NC(=O)[C@H](CCC(N)=O)NC(=O)[C@@H](NC(=O)[C@H](CC(=O)O)NC(=O)CNC(C)=O)C(C)C)C(=O)N[C@@H](CCC(=O)O)B(O)O] | 70 ± 11 |
| 7 | 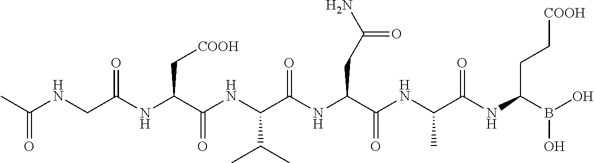 [SMILES: CC(=O)NCC(=O)N[C@@H](CC(=O)O)C(=O)N[C@H](C(=O)N[C@@H](CC(N)=O)C(=O)N[C@@H](C)C(=O)N[C@@H](CCC(=O)O)B(O)O)C(C)C] | 37 ± 3 |
| 8 | 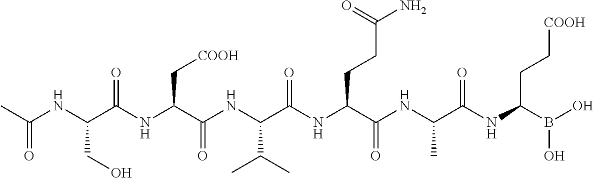 [SMILES: CC(=O)N[C@@H](CO)C(=O)N[C@@H](CC(=O)O)C(=O)N[C@H](C(=O)N[C@@H](CCC(N)=O)C(=O)N[C@@H](C)C(=O)N[C@@H](CCC(=O)O)B(O)O)C(C)C] | 40 ± 3 |
| 9 | 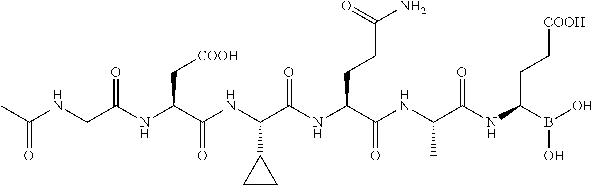 [SMILES: CC(=O)NCC(=O)N[C@@H](CC(=O)O)C(=O)N[C@H](C(=O)N[C@@H](CCC(N)=O)C(=O)N[C@@H](C)C(=O)N[C@@H](CCC(=O)O)B(O)O)C1CC1] | 12.5 ± 1.4 |
| 10 | 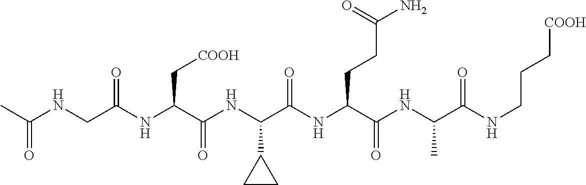 [SMILES: CC(=O)NCC(=O)N[C@@H](CC(=O)O)C(=O)N[C@H](C(=O)N[C@@H](CCC(N)=O)C(=O)N[C@@H](C)C(=O)NCCCC(=O)O)C1CC1] | (1.4 ± 0.3) × 10³ |

### Substrate and Inhibitor Activation of CylA Activity at Low Concentrations.

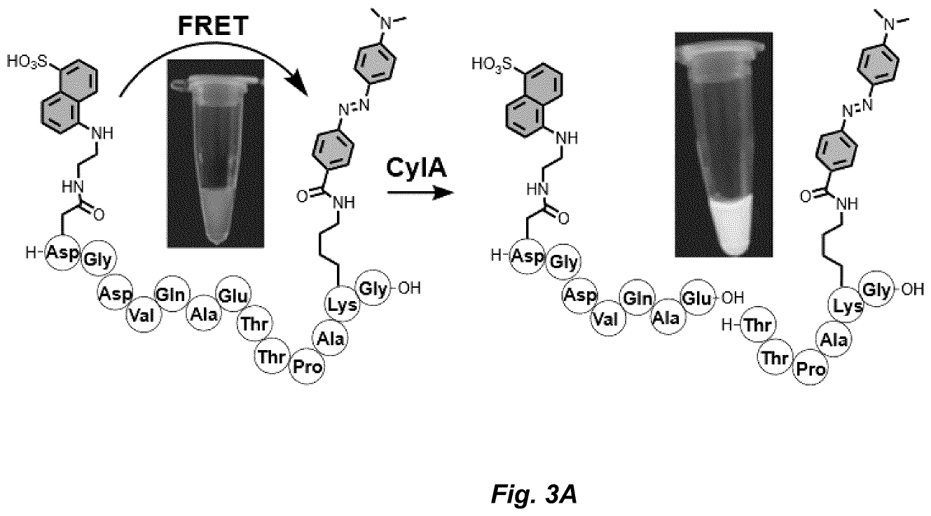

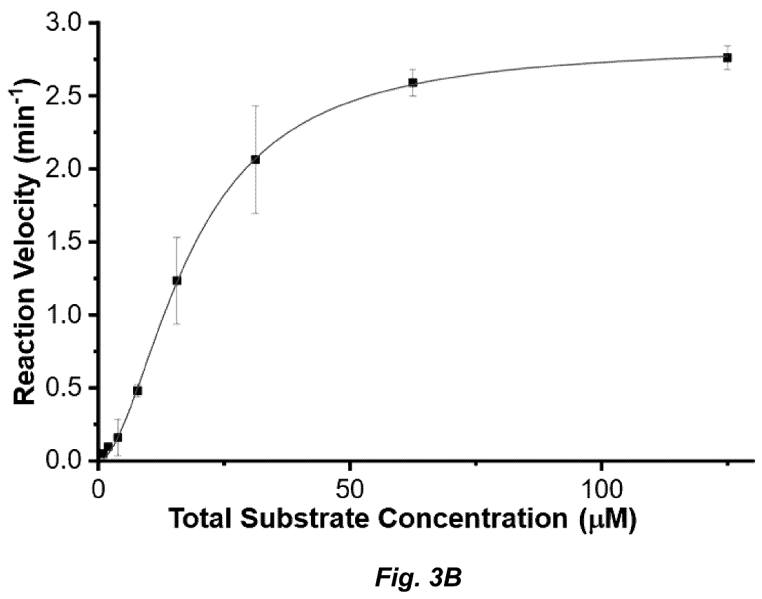

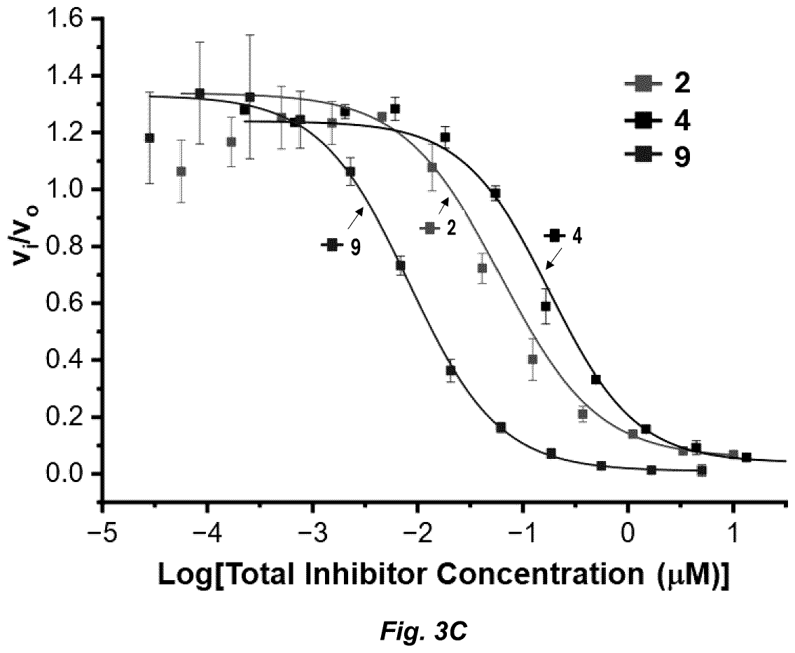

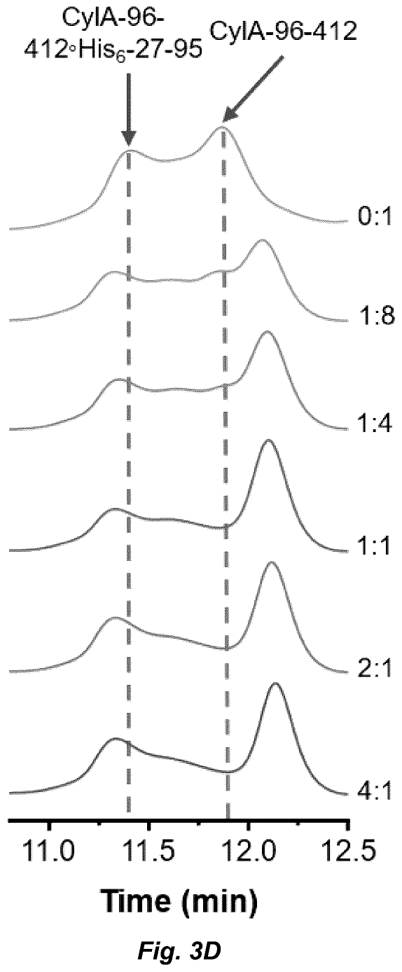

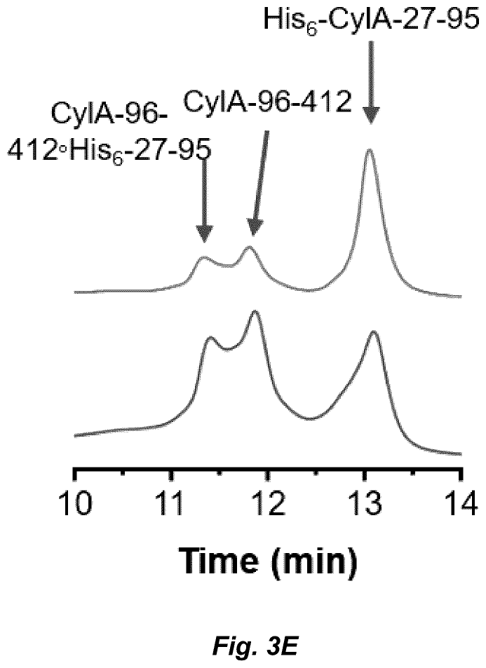

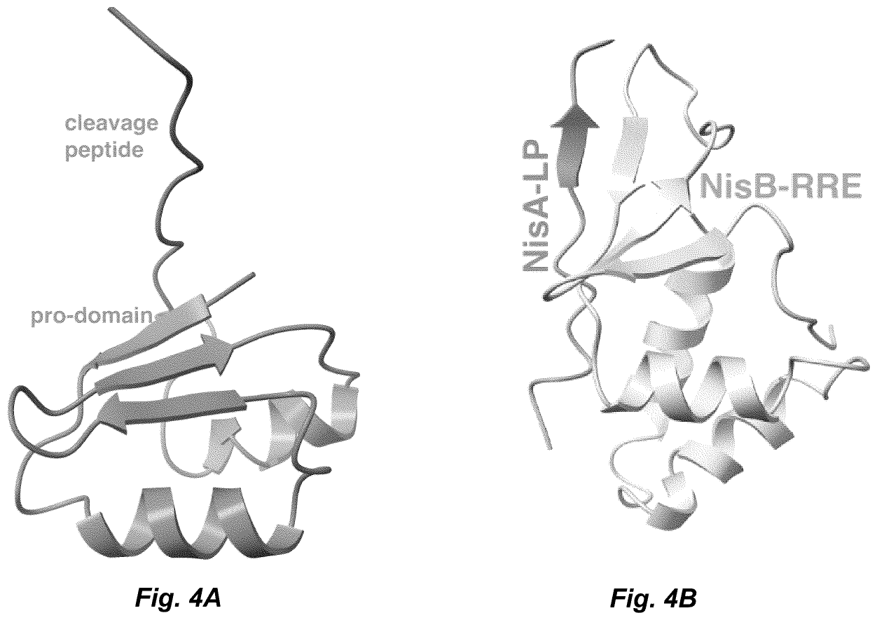

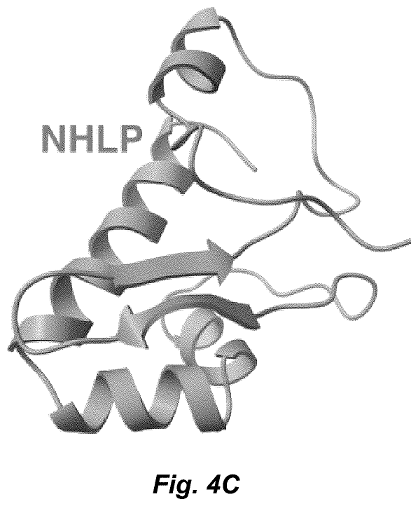

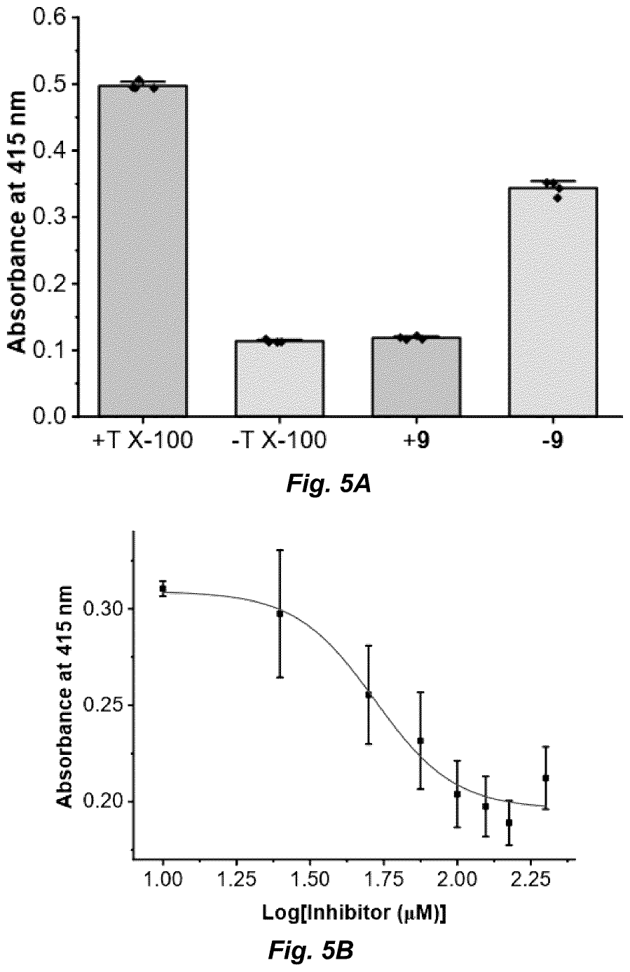

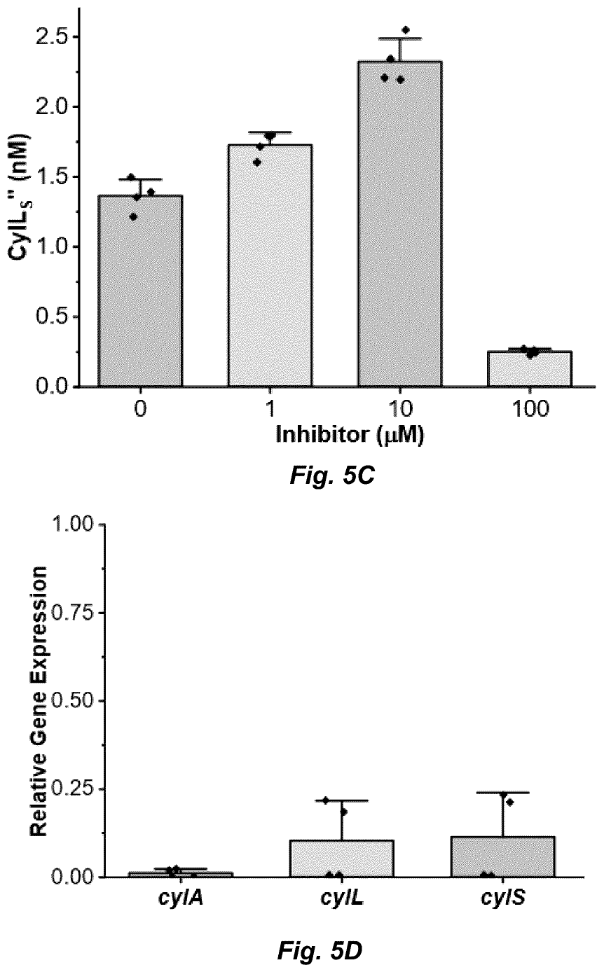

A FRET assay was developed to directly detect the inhibition of His₆-CylA-27-412 activity by inhibitor 9. To prepare a FRET labelled substrate, the substrate recognition sequence and a portion of the N-terminus of unmodified CylL_{S} was embedded in between N-terminal Asp(EDANS) and C-terminal Lys(DABCYL) residues using Fmoc SPPS (FIG. 3A). The FRET labelled substrate was incubated with nanomolar concentrations of His₆-CylA-27-412 at 37° C. overnight. High resolution LCMS-MS analysis revealed that the peptide was cleaved under these conditions producing fragments consistent with cleavage at the C-terminus of Glu (See Moreira et al., Supplementary FIGS. 4 and 5). Visually, a sample of the FRET peptide treated with His₆-CylA-27-412 was significantly more fluorescent than a CylA-free control (FIG. 3A) showing that the peptide worked as designed.

The FRET peptide was used to continuously monitor the activity of His₆-CylA-27-412 at different substrate concentrations. Plotting the changes in fluorescence emission intensity as a function of total substrate concentration yielded a sigmoidal curve indicative of substrate activation (FIG. 3B). Fitting this curve to the Hill form of the Michaelis-Menten equation gave a Km equal to 18.3±1.4 μM and a Hill co-efficient of 1.8+0.2, suggesting significant activation of CylA by the FRET peptide substrate. Since the FRET peptide is based on the natural substrate of CylA, it was hypothesized that mCylL_{S}, mCylL_{L} and/or a mixture of mCylL_{S}:mCylL_{L} could also activate His₆-CylA-27-412. Treating samples of His₆-CylA-27-412 with these pre-cytolysin peptides increased the initial rate of FRET peptide cleavage by a factor of up to 1.5±0.3 (See Moreira et al., Supplementary FIG. 6) at total peptide concentrations in the nanomolar range but above the total concentration of CylA. In contrast, micromolar concentrations of pre-cytolysin peptides lowered the rate of FRET peptide proteolysis suggesting that the pre-cytolysin peptides are competing for the same active site as the FRET substrate under these conditions. Overall, these results are consistent with the natural substrate activating CylA.

Treating His₆-CylA-27-412 with different concentrations of compound 9 showed inhibition with an IC₅₀ of 8.3±0.6 nM (FIG. 3C). However, similar to the activation by substrate at low concentration, compound 9 activated His₆-CylA-27-412 at substoichiometric concentrations (the total enzyme concentration was 20 nM). When His₆-CylA-27-412 was treated with 762, 254, or 85 or 28 μM of 9, the rate of FRET peptide cleavage increased by a factor of up to 1.3±0.2 relative to an uninhibited control. To investigate if this effect was specific for 9, the assay was conducted with 2 and 4. Similar to 9, 2 and 4 functioned as inhibitors at higher concentrations giving IC₅₀ values equal to 61+17 nM and 178+15 nM (FIG. 3C), respectively, and activated the enzyme at lower concentrations. Inhibitor 2 activated His₆-CylA-27-412 at 4.6 to 0.06 nM and inhibitor 3 activated the enzyme at 18.3 to 0.22 nM. All three inhibitors activated His₆-CylA-27-412 by a maximum factor of approximately 1.3. These data suggest that activation of His₆-CylA-27-412 occurs under conditions where inhibitor binding is competent, but active site occupancy is low.

Substoichiometric activation of a serine protease by a peptidic boronic acid inhibitor based on the natural substrate sequence of the enzyme has been characterized for DegP and HTRA1, which are conformation-specific protease/chaperones. Detailed biophysical studies on the activation of DegP by substoichiometric quantities of peptidic boronic acid inhibitor revealed that binding of the inhibitor to the active site of a protomer increased the activity of adjacent protomers in the resting hexameric state, while stoichiometric quantities of the inhibitor produced an inactive dodecameric form. Both DegP and HTRA1 are S1 proteases that differ structurally from CylA, raising questions about the mechanism of substoichiometric activation of CylA by compound 9. To our knowledge, substrate activation and substoichiometric activation by a substrate-based boronic inhibitor have not been reported for S9 subtilisins. Enzymes of this class undergo maturation through a general mechanism in which the rate determining step is often dissociation of the N-terminal pro-domain from the enzyme active site. It is possible that release of the CylA pro-domain from the C-terminal catalytic domain is accelerated by substrate binding, or by binding substrate-like species, like inhibitor 9. If the pro-domain is slow to reassociate with the C-terminal catalytic domain due to unfolding upon displacement, an increase in CylA activity could be observed at inhibitor concentrations that rapidly equilibrate with the enzyme active site, but give marginal active site occupancy. Alternatively or simultaneously, the increase in activity could follow a DegP-like mechanism in which inhibitor binding influences oligomeric states. To gain more insight into this phenomenon, His₆-CylA-27-412 that had undergone complete autolytic cleavage as determined by SDS-PAGE and that had been purified by size exclusion chromatography (SEC), was analyzed by analytical SEC (aSEC) in the presence of inhibitor 9. The aSEC trace showed two peaks which had retention times that corresponded to masses consistent with CylA-96-412 with and without its pro-domain non-covalently attached (FIG. 3D). The addition of substoichiometric quantities of compound 9 (1:8 9: CylA) increased the complexity of the trace in the region where CylA-96-412 and CylA-96-412 non-covalently bound to its pro-domain (CylA-96-412·His₆-27-95) elute when 9 is not present. Although several overlapping peaks seem to have formed, two pronounced peaks on each end of the 10.5-12.5 min grouping can be observed (FIG. 3D).

The peak eluting at 12.1 min has a retention time that is slightly longer than CylA-96-412 (11.9 min) and the peak eluting at 11.3 min has a retention time that is slightly shorter than CylA-96-412·His₆-27-95 (11.4 min). Adding more inhibitor relative to enzyme increased the intensity of the peak at 12.1 min relative to the CylA-96-412·His₆-27-95 signal and the transition appears complete once a stoichiometric equivalent of inhibitor is added (FIG. 3D). When a similarly prepared sample of His₆-CylA-27-412 was treated with mCylL_{S}, mCylL_{L} or a 1:1 mixture of mCylL_{S} and mCylL_{L} for 10 min at room temperature and then analyzed by aSEC, a relative decrease in the amount of CylA-96-412·His₆-27-95 was observed (See Moreira et al., Supplementary FIG. 7). SEC retention time data is inconsistent with CylA forming higher order oligomers, ruling out a DegP-like activation mechanism. Instead, the SEC data is consistent with inhibitor or substrate binding to both CylA-96-412 and CylA-96-412·His₆-27-95 and encouraging but not necessitating pro-domain displacement from CylA-96-412His₆-27-95.

To assess whether pro-domain displacement is reversible, the isolated pro-domain was incubated with a sample of SEC-purified His₆-CylA-27-412 for 20 min at room temperature, and then analyzed by aSEC (FIG. 3E). No change in the ratio of CylA-96-412:CylA-96-412·His₆-27-95 was observed demonstrating that once the pro-domain is released from the C-terminal domain it cannot readily rebind. Analysis of the pro-domain isolated during the SEC purification of His₆-CylA-27-412 using circular dichroism (CD) showed it was in an unfolded state (See Moreira et al., Supplementary FIG. 8). These results are consistent with pro-domain folding being essential for and dependent on interactions with the C-terminal domain which is typical for related proteases.

Collectively, these observations are consistent with binding of inhibitor or substrate encouraging irreversible release of the pro-domain resulting in an increase in the population of CylA-96-412 relative to CylA-96-412·His₆-27-95 and increasing enzyme activity. The increase in enzyme activity was small (1.3-1.5-fold) in most cases probably because CylA-96-412 is already present in the sample and CylA-96-412·His₆-27-95, which can bind to the inhibitor, may have some proteolytic activity. Inhibitor and substrate activation suggests that CylA may release its pro-domain more rapidly when substrate concentrations begin to increase, possibly protecting the natural producer from the proteolytic CylA until substrate concentration surpasses a threshold level. This may provide another point of control for cytolysin biosynthesis and prevent triggering of the CylR1/CylR2 response at lower cell densities when substrate expression is at basal levels. Importantly, aSEC results (FIG. 3D), in combination with dose-response studies (FIG. 3C), demonstrate that inhibitor 9 can inhibit all active forms of CylA at low nanomolar concentrations.

### The Structure of the CylA Pro-Domain-Catalytic Domain Complex.

To afford a high-resolution structure, full-length CylA (residues Leu27-Lys412, lacking the N-terminal secretion signal sequence) with an N-terminal polyhistidine tag was expressed in E. coli, refolded from the insoluble fraction and incubated in situ to allow for autoproteolytic processing. Following near-complete auto-processing, as determined by SDS-PAGE analysis, the soluble, folded CylA-96-412·His₆-27-95 complex was purified by nickel affinity chromatography. The cleaved domains co-eluted as a single species suggesting that the pro-domain was tightly bound to the protease domain. Following size-exclusion chromatography, diffraction quality crystals of the complex were obtained using the vapor diffusion method. Crystals of CylA-96-412 His₆-27-95 diffracted to a Bragg limit of 1.3 Å and initial crystallographic phases were determined using an AlphaFold model (Jumper et al. Nature, 596, 583-589 (2021).) of the catalytic domain as a molecular replacement search probe. The ~75-residue pro-domain was built using a combination of automated and manual methods into electron density maps calculated using initial phases calculated from the catalytic domain.

The crystal structure of CylA-96-412·His₆-27-95 reveals the complex between the catalytic domain and the proteolytically excised pro-domain (See Moreira, FIG. 6A). The active site of the catalytic domain, consisting of the catalytic triad residues Asp142, His173, and Ser359, is additionally occupied by the C-terminal tail of the pro-domain ending with the α-carboxylate of residue Glu95 (position −1 for autoproteolysis), which is located 3.2 Å away from the nucleophilic Ser359 side chain oxygen. The side chain of Asn263 and the backbone amide of Thr275 are positioned to provide an oxyanion hole to stabilize the negative charge accumulated in the transition state. Clear and continuous electron density is evident for the entirety of the pro-domain tail detailing interactions with the PDFKPE (SEQ ID NO: 12) recognition sequence preceding the scissile bond. Pairwise comparison of the CylA protease domain against the Protein Data Bank, using the DALI server, shows an overall conservation with other subtilisin-like lanthipeptide proteases such as LicP (PDB Code 4ZOQ; Z-score=42.9, RMSD of 2.0 Å over 302 aligned Ca residues), the isolated protease domain NisP⁵⁵ (PDB Code 4MZD; Z-score-35.6, RMSD of 2.3 Å over 280 aligned Ca residues), and the as of yet uncharacterized homolog of EpiP (PDB Code 3QFH; Z-score=35.5, RMSD of 2.4 Å over 275 aligned Cα residues). The CylA structure is also related to other canonical S9 proteases, including various homologs of subtilisin.

A closer examination of the active site shows interactions with the pro-domain PDFKPE (SEQ ID NO: 12)C-terminal sequence that account for substrate specificity (See Moreira, FIG. 6B). The side chain of Glu95 at the −1 position is within hydrogen bonding distance with the side chains of Ser260, His275 and the backbone amide of Gly262. The sidechain of Glu264 is outside hydrogen bonding distance to Glu95 (4.4 Å) but may be involved in electrostatic stabilization of protonated His275. The residues at positions −2 (Pro94) and −3 (Lys93) face away from the catalytic domain, accounting for the lack of conservation at these sites between the pro-domain and the GDVQAE (SEQ ID NO: 13) specificity sequence in the CylL_{L}′ and CylL_{S}′ substrate peptides.

Notably, the −4 residue (Phe92) is directed into the catalytic domain where it is engaged in van der Waals contacts with residues in a pocket composed of Val195, Met196, Met206, and Leu225. These interactions provide a rationale for the preference of a hydrophobic residue (Val or Phe) at the −4 position in both the self-cleavage and in the substrate peptide of CylA, as well as in the related LicP protease. Lastly, Asp91 at the −5 position is directed away from the catalytic domain but is located within hydrogen bonding distance of Asp200 and Asn202. These observations suggest that CylA should show higher specificity at −1 and lower specificity at the −5 position, which contradicts the data presented in Table 1, and suggests that the substrate recognition site of CylA undergoes some restructuring upon release of the pro-domain and/or upon substrate binding. Despite much effort, we were not able to obtain a structure of an inhibitor bound to the catalytic domain.

To gain more insight into the hypothesized restructuring, we used ColabFold v1.5.5 to generate a structure of CylA-96-412 with GDVQAE (SEQ ID NO: 13) bound to the substrate recognition site (See Moreira et al., Supplementary FIG. 9). The structure generated by ColabFold shows GDVQAE (SEQ ID NO: 13) bound to the substrate recognition near the active site and CylA-96-412 aligns well with the crystal structure (RMSD=0.5 Å over 386 aligned Cα residues); however, deviations are concentrated at a location near the substrate recognition groove. Notably, in the ColabFold structure, a short, anti-parallel B-sheet located near the substrate binding groove orients the side chain of Lys229 towards Asp-5 generating a salt bridge. In the crystal structure, this region does not contain the anti-parallel B-sheet and the Lys229 ε-NH₂ is 12.4 Å from the Asp-5 carboxylate. These observations suggest that this region of CylA may be dynamic, undergoing spontaneous re-structuring in solution or in response to pro-domain release and/or substrate binding. The latter hypothesis is consistent with the observed activation of CylA when it is treated with substrate or substoichiometric concentrations of inhibitor.

A structure-based DALI search against the PDB using the coordinates of only the pro-domain revealed structural similarities that may be instructive. As expected, the CylA pro-domain (FIG. 4A) shares structural similarity with the pro-domain of subtilisin BPN′ (PDB Code 1SPB, Z-score=3.9, RMSD of 3.3 Å over 58 aligned Cα residues). However, structural similarity is also detected with the RiPP recognition element (RRE) domain of NisB (PDB Code 4WD9, Z-score=4.6, RMSD of 3.0 Å over 62 aligned Cα residues; FIG. 4B). Weaker structural resemblance is also noted between the CylA pro-domain, and the nitrile hydratase-like protein (NHLP) type LPs found in proteusin biosynthetic pathways (FIG. 4C). The RRE, NHLP and the protease pro-domains interact with and stabilize their cognate catalytic domain in the active form. Notably, cytolysin is a product of a class II lanthipeptide pathway and none of the biosynthetic enzymes employ either an RRE or a NHLP peptide. Hence, the structural similarity of the CylA pro-domain with domains involved in the biosynthesis of other classes of RiPPs represents a convergent strategy for engaging catalytic domains with their substrates.

Compound 9 Inhibits Toxin Maturation in C+ E. faecalis.

Inhibitor 9 showed promising activity in vitro suggesting that it may effectively prevent toxin production in C+ E. faecalis. Using the antiSMASH program, we identified a commercially available strain of C+ E. faecalis (E. faecalis ATCC 29212) that produces a cytolysin congener which differs from the originally characterized cytolysin at one amino acid residue in CylL_{L}″. The BGC containing this cytolysin congener possessed genes encoding homologs of CylR1/R2, CylM, CylB, CylI and CylA. The CylA sequence contained in this BGC was 99% identical to the previously reported sequence for CylA. Initial experiments confirmed that this strain is hemolytic at high cell densities (OD₆₀₀≥1), like the previously reported C+ E. faecalis strain.

To determine whether the inhibitor can block hemolysis, an overnight culture of C+ E. faecalis was diluted to a desired cell density, combined with washed rabbit erythrocytes, then deposited into a 96 well plate. Wells containing C+ E. faecalis showed significant hemolysis over a 4-hour period at 37° C. when a relatively dense inoculum was used (OD₆₀₀=0.35). To quantify this observation, hemoglobin absorbance of the supernatant was measured (FIG. 5A). Wells not containing bacteria but containing erythrocytes were used as controls to evaluate erythrocyte stability over the course of the assay. When Triton X-100 (T X-100) was added to one of these wells, which rapidly lyses erythrocytes, the absorbance at 415 nm increased 5-fold (FIG. 5A). Similarly, incubating C+ E. faecalis in wells containing erythrocytes increased absorbance at 415 nm 4-fold relative to the negative control (FIG. 5A).

In contrast, adding inhibitor 9 to wells containing C+ E. faecalis and erythrocytes completely halted hemolysis (FIG. 5A). This assay was conducted over a range of inhibitor concentrations and an EC₅₀ equal to 52+8 μM was determined (FIG. 5B). Attenuation of hemolytic activity was not caused by toxicity of 9 to E. faecalis because 9 did not impact the growth of C+E. faecalis at 100 μM (See Moreira et al., Supplementary FIG. 10). Furthermore, toxicity studies involving HeLa cells found inhibitors 2, 3, 5-9 to be non-toxic at concentrations≤100 μM suggesting protease selectivity and potential utility for in vivo studies (See Moreira et al., Supplementary FIG. 11).

To determine if inhibitor 9 can block the maturation of cytolysin in C+ E. faecalis, an

LCMS method was developed to estimate the concentration of CylL_{S}″ in extracts of C+ E. faecalis. This method employed biosynthetically prepared ¹⁵N,¹³C-labelled CylL_{S}″ as an internal standard. Cultures of E. faecalis were treated with inhibitor 9 or vehicle then lyophilized and extracted with methanol. Cell samples treated with 1 or 10 μM of 9 produced similar or significantly more CylL_{S}″ relative to samples that were not treated with inhibitor 9 (FIG. 5C).

In contrast, cells treated with 100 μM inhibitor 9 produced quantities of CylL_{S}″ that were on average 5.5-fold less than the untreated control. These experiments demonstrate that inhibitor 9 can slow the production of cytolysin, but bidirectional modulation is possible. The increase in the amount of extractable CylL_{S}″ observed in samples treated with 1 or 10 μM inhibitor 9 compared to untreated samples is intriguing (FIG. 5C). Based on the data presented in FIGS. 4C-E and Moreira et al., Supplementary FIG. 16, it is tempting to conclude that these increases are due to substoichiometric activation of CylA by inhibitor 9; however, without a detailed analysis of the amount of active CylA relative to amount of inhibitor and precise knowledge of the relative populations of the states of CylA over the course of the experiment, it is difficult to conclude that this effect is manifesting in these experiments. Regardless of mechanism, the ability to bidirectionally modulate toxin production may prove useful for in vivo studies and could provide details on the role of cytolysin in diverse bacterial communities.

Finally, to see if CylA inhibition results in a reduction of the transcription of the cytolysin BGC, RT-qPCR was performed on samples of total RNA extracted from overnight cultures of C+E. faecalis. Since the cytolysin BGC is often expressed in two transcripts due to the presence of a weak intergenic terminator between cylL and cylM, primers and amplicons were selected for cylL, cylS and cylA. Compared to an untreated control, treatment of E. faecalis with 9 (100 μM) resulted in an 86-fold reduction in cylA transcripts and a 9-10 fold reduction in cylL and cylS transcripts (FIG. 5D). These results demonstrate that, in addition to preventing toxin maturation, CylA inhibition strongly reduces the transcription of the cytolysin BGC, as predicted by the model in FIG. 1 of Moreira et al.

The intersection between the human microbiome, human health and natural products has added an exciting dimension to natural products research. At this interface, the biosynthetic information amassed from extensive study of natural products biosynthesis finds a new application that can directly impact human health. In this work, knowledge of the cytolysin BGC was used to rationally develop small molecule inhibitors against this deadly virulence factor, which directly contributes to high mortality in patients that host C+ E. faecalis and have alcoholic hepatitis. Targeting a key protease that is essential for toxin activation, substrate-based inhibitors were developed that had low micromolar to low nanomolar activities depending on the experimental context. Studies on the inhibition of CylA using these inhibitors revealed that CylA is a substrate-activated protease and this feature provides an additional potential point of control for cytolysin production in natural settings. A crystal structure of the CylA catalytic domain non-covalently bound to its pro-domain, in combination with inhibitor-enzyme structure-activity studies, provides a detailed description of substrate recognition. Finally, bioactivity studies demonstrated that inhibitors of CylA can block the hemolytic activity of C+ E. faecalis through preventing toxin maturation and attenuating expression of the cytolysin BGC.

In addition to providing a potential lead for the development of a prophylactic for cytolysin-induced disease states and/or treatment of those states, in vivo studies using these inhibitors may provide an avenue to better understand the mechanisms of cytolysin induced disease states and the impact of cytolysin on the bacterial communities found in healthy hosts. Studies of this nature could further broaden understanding of the clinical impact of cytolysin and bring to light a deeper connection between C+ bacteria and human health.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

All reagents used for chemical synthesis were purchased from Sigma Aldrich, Chem-Impex and CombiBlocks. N,N-Dimethylformamide (DMF) and dichloromethane (DCM) were dried over 3 Å molecular sieves before use. Chromatography was performed using 60 Å silica gel. Compound 13 was prepared as described in Wünsch et al., Beilstein J. Org. Chem. 13, 2428-2441 (2017). Compounds 14, 16 and 18 were prepared following a previously described in Fini et al., Org. Biomol. Chem. 19, 6687-6691 (2021). ¹H and ¹³C NMR spectra were collected using at 500 and 125 MHz, respectively on a Bruker spectrophotometer. Spectra were referenced to the residual chloroform solvent peak (7.26 ppm, ¹H; 77.0 ppm, ¹³C). Matrix-assisted laser desorption/ionization time-of-flight mass spectrometry (MALDI-TOF MS) analysis was performed on a Bruker UltrafleXtreme MALDI-TOF mass spectrometer (Bruker Daltonics) at the University of Illinois School of Chemical Sciences Mass Spectrometry Facility. High resolution LCMS analysis was performed using an Agilent 1290 LC-MS QToF instrument. LC separations were conducted using an Kinetex®, 2.6 μm, 150×2.1 mm column. Tandem MS-MS fragmentation was conducted using normalized collision energies of 20 and 30. Fluorescence intensities and absorbances were measured using a Biotek Synergy H₄. Circular dichroism data was collected using a Jasco J-1500 spectrophotometer. Overnight cultures were prepared from colonies of Enterococcus faecalis ATCC 29212 grown on brain heart infusion (BHI) agar that were grown overnight at 37° C. in BHI. pRSFDuet-1/CylA-27-412 was prepared as described in Tang et al, J. Ind. Microbiol. Biotechnol. 46, 537-549 (2019). Escherichia coli was cultured in lysogeny broth (LB) prepared according to the Miller formula.

### tert-Butyl (R)-4-(((R)-tert-butylsulfinyl)amino)-4-(4,4,5,5-tetramethyl-1,3,2dioxaborolan-2-yl) butanoate (15)

A flask was charged with tricyclohexylphosphine tetrafluoroborate, toluene (0.8 mL), aqueous CuSO₄ (1.22 mL of a 30 mM solution; 1.2 mol %) and benzylamine (16.8 μL, 0.152 mmol, 5.0 mol %) and the contents were stirred at room temperature for 10 min before adding a solution of 13 (800 mg, 3.06 mmol, 1.0 equiv.) dissolved in toluene (7.2 mL). To this mixture, was added bis(pinacolato)diboron (1.55 g, 6.12 mmol, 2.0) and the resulting suspension was stirred at room temperature overnight. The reaction mixture was diluted in ethyl acetate (50 mL) and filtered through a plug of deactivated silica gel (100:35 silica: water) eluting with ethyl acetate. The filtrate was concentrated via rotorary evaporation and the residue was loaded onto a deactivated silica gel column pre-equilibrated with DCM. A gradient of 100% DCM to 50% DCM/50% ethyl acetate was used. The elute 15 was detected using KMnO₄ stain. This procedure yielded 15 as a colorless oil (655 mg, 1.68 mmol, 55% yield). ¹H NMR (300 MHz, CDCl₃) δ 3.36 (d, J=6.8 Hz, ¹H), 3.06 (q, J=6.9 Hz, ¹H), 2.39 (t, J=7.8 Hz, 2H), 1.96 (m, 2H), 1.45 (s, 9H), 1.29 (m, 12H), 1.21 (s, 9H). ¹³C NMR (125 MHz, CDCl₃) δ 172.95, 84.34, 80.47, 56.37, 55.67, 33.05, 28.33, 25.24, 25.15, 24.76, 22.77.

### (R)-4-(tert-butoxy)-4-oxo-1-(4,4,5,5-tetramethyl-1,3,2-dioxaborolan-2-yl) butan-1-aminium chloride (17)

Compound 15 (65 mg, 0.167 mmol, 1 equiv.) was dissolved in 1,4-dioxane (0.83 mL) and to this solution was added methanol (69 μL, 1.67 mmol, 10 equiv.) followed by HCl (4.0 M solution in 1,4-dioxane, 67 μL, 0.267 mmol of HCl, 1.7 equiv.). The resulting mixture was stirred at room temperature for 1 h before dilution with toluene (1 mL) and concentrating to dryness. The crude residue was immediately used in the synthesis of 1-10.

### General Fmoc Solid-Phase Peptide Synthesis Conditions.

Fmoc SPPS was performed using a Liberty Blue™ peptide synthesizer (CEM). All resins were purchased preloaded from Chem-Impex and loading was determined immediately before starting the synthesis. All steps were performed at room temperature. Fmoc removal was performed by treating resin bound Fmoc protected peptide with 20% piperidine/DMF (3×10 min) followed by washing the resin with DMF (6×1 min). Fmoc-protected amino acids were coupled to the solid phase by mixing the resin with a solution containing Fmoc protected amino acid (3 equiv.), ethyl cyanohydroxyiminoacetate (3 equiv.) and N,N′-diisopropylcarbodiimide (DIC) (3 quiv.) in DMF for 1 h before draining and washing the resin with DMF (6×1 min). This process was repeated until the final amino acid was coupled. When desired, N-terminal acetylation was performed by removing the Fmoc group from the N-terminus then treating the resin bound peptide with resin capping solution (90:8:2 DMF: acetic anhydride: DIPEA) for 10 min. The resin was washed with DMF (6×1 min) then DCM (6×1 min).

Synthesis of 1-10 Starting from 1a-9a.

Resin bound precursor peptides 1a-9a were treated with 1% TFA/DCM for 20 min to cleave the peptide from the solid support. The resin was removed by filtration and washed with additional 1% TFA/DCM. Solvent was removed via rotorary evaporation and the residue was dissolved in 1:1 DCM: toluene and concentrated again to remove trace TFA. For the synthesis of 1b, 2b and 4b-9b, DIPEA (3.0 equiv.) was added to solutions of freshly prepared 17 (1.3 equiv.) in dry DCM (final concentration of 1a-9a was 0.05 M) and this solution was transferred to a 1 dram vial containing 1a, 2a or 4a-9a and HATU (1.2 equiv.) was added. For the synthesis of 3b, the same reactions were setup using the same conditions but 18 was used in place of 17. In all cases, the formed suspension was stirred for 13 h at room temperature, then diluted 10-fold with DCM and washed an equal volume of water. The organic layer was dried over sodium sulfate and concentrated. The crude peptide residue was dissolved in cleavage cocktail (TFA:triisopropylsilane:H₂O 95:2.5:2.5) to a concentration of 18 mg of crude peptide per milliliter. The solution was stirred for 1 h at room temperature then concentrated with a stream of N₂. Peptide was precipitated from the concentrated residue through the addition of ice cold diethyl ether and the precipitate was pelleted by centrifugation (4000 g for 10 min) then washed with another volume of diethyl ether. The peptide residue was dissolved in water and loaded onto a Bond Elut C18 Solid Phase Extraction (SPE) Cartridge. The stationary phase was washed with H₂O+0.1% TFA then the peptide was eluted with 60% CH₃CN+0.1% TFA/40% H₂O+0.1% TFA. The elution fraction was lyophilized and the lyophilized peptide was dissolved in water prior to purification.

### Purification of Peptides 1-10.

Crude peptides 1-10 were purified by reversed-phase UHPLC using a ThermoFisher Vanquish UHPLC using an Accucore™ C18, 2.6 μm, 150×4.6 mm column and a flow rate of 0.4 mL/min. Solvent A was H₂O+0.1% trifluoroacetic acid (TFA) and solvent B was CH₃CN+0.1% TFA. The same gradient method was used to purify each peptide: 2% solvent B for 2 min followed by a linear ramp to 20% solvent B over 8 min. Fractions containing pure peptide were determined by LCMS and combined and concentrated by lyophilization. Lyophilized peptide was dissolved in H₂O and characterized by high-resolution LCMS (See Moreira et al., Supplementary FIGS. 1 and 2). The stock concentration of 1 was determined gravimetrically, the concentrations of stocks of 2-10 were determined by comparing the absorbance of each sample at 205 nm to the absorbance of 1. Absorbances at 205 nm were determined from the peak areas of traces collected using the UV-detector equipped to the LCMS. For peptide 2, additional purification was necessary.

After UHPLC, crude 2 (20 mg) was dissolved in dry DMF (2 mL) and incubated with 1-glycerol resin (200 mg, 1.1 mmol/g) that was pre-swelled in DMF. The mixture was incubated for 13 h at room temperature then the resin was filtered off and washed with dry DMF (6×1 min). The resin was treated with 2 mL of elution cocktail (DCM:methanol:H₂O=5:4:1) for 3×30 min and the combined elutions were concentrated via rotary evaporation. The crude residue was dissolved in H₂O and lyophilized. The lyophilized peptide 2 was characterized by high-resolution LCMS (Table 2). Stock concentrations were measured by LCMS through comparison to a standard using absorbance at 205 nm.

### Synthesis and Purification of the FRET Substrate Peptide.

The synthesis used Fmoc-L-Gly-Wang loaded polystyrene resin, which was extended to the penultimate residue (Gly) using standard Fmoc SPPS conditions. Fmoc-L-Asp(EDANS)-OH was coupled to the N-terminus by incubating the resin bound peptide with Fmoc-L-Asp(EDANS)-OH (3 equiv.), benzotriazolyloxy-tris [pyrrolidino]-phosphonium hexafluorophosphate (3 equiv.) and (DIPEA; 3 equiv.) dissolved in dry DMF at room temperature for 13 h. The resin was washed with DMF (6×1 min) then DCM (6×1 min), and the peptide was cleaved from the resin and globally deprotected through treatment with cleavage cocktail (TFA:phenol:H₂O 90:5:5) for 90 min at room temperature. TFA was removed under a stream of N₂ and the crude peptide was precipitated from this residue through the addition of ice-cold diethyl ether. The precipitated peptide was pelleted by centrifugation (4000 g for 10 min) then washed with another volume of cold diethyl ether. The pelleted peptide was dissolved in 1:1 H₂O:CH₃CN and purified by semi-preparative R-HPLC using a Macherey-Nagel NUCLEODUR C18 HTec, 10 μm, 250×10 mm column. Solvent A was H₂O+0.1% TFA and solvent B was CH₃CN+0.1% TFA. A gradient method was used which started with 2% solvent B for 10 min followed by a linear ramp from 2% to 80% solvent B over 25 min and then another linear ramp from 80% to 100% solvent B. Fractions were analyzed by MALDI-TOF MS and those containing pure peptide were combined and lyophilized. Lyophilized peptide was dissolved in 1:1 CH₃CN: H₂O and characterized by LCMS-MS (See Moreira et al., Supplementary FIG. 4). FRET peptide concentration was determined by the absorbance of DABCYL at 482 nm using an extinction coefficient of 25500 cm⁻¹ M⁻¹.

### Expression and Purification of His₆-CylA-27-412.

Escherichia coli BL21 (DE3) cells were transformed with pRSFDuet-1/CylA-27-412 and plated on LB containing 50 mg/L kanamycin. A single colony was picked and used to inoculate 20 mL of LB containing 50 mg/L kanamycin which was incubated overnight at 37° C. The overnight culture was used to inoculate 1 L of LB (1:100) which was incubated at 37° C. until an OD₆₀₀ of 0.8 was reached. The culture was then cooled on ice for 30 min and IPTG was added to a final concentration of 250 μM. The flask was incubated at 18° C. for 16 h before collecting the cells by centrifugation (6000 g for 15 min). The cell pellet was resuspended in CylA lysis buffer (20 mM HEPES, 1 M NaCl, 10% glycerol, pH=7.5) and the cells were lysed by sonication (8 rounds of the 30 s on followed by 30 s off). The cell lysate was centrifuged (120 k g for 45 min) and the pellet was resuspended in Pellet extraction buffer (8 M urea, 1 M NaCl, 20 mM HEPES, 10% glycerol, pH=7.5). The pellet suspension was sonicated (7 rounds of the 30 s on followed by 30 s off) then centrifuged (50000 g for 45 min). The supernatant was collected and dialysed against Dialysis buffer (20 mM HEPES, 300 mM NaCl, pH=7.5) overnight. Precipitate was removed by centrifugation (120 k g for 45 min) and the supernatant was subjected to immobilized metal affinity chromatography (IMAC) using Ni-resin as described previously⁴⁴. Elution fractions containing His₆-CylA-27-412 were combined and dialyzed against Dialysis buffer. The IMAC purified protein was incubated at 4° C. until SDS-PAGE showed that the sample underwent autolysis (usually 48 h). His₆-CylA-27-412 was further purified by size exclusion chromatography (SEC) using a Superdex 75 column (Cytiva). Fractions containing His₆-CylA-27-412 were concentrated via 3-kDa MWCO centrifugal filter.

### Crystallization and Data Collection of His₆-CylA-27-412.

The concentrated protein from the size exclusion chromatography was used for crystallization by sitting drop vapor diffusion method. His₆-CylA-27-412 purified by size exclusion chromatography was concentrated to 15.43 mg/ml and mixed in 1:1 ratio (v/v) with a solution of 0.2M ammonium sulfate, 0.1 M sodium acetate trihydrate pH 4.2, 22% (w/v) polyethylene glycol 2000 monomethyl ether and incubated against the latter solution using the hanging drop vapor diffusion method. Large rhombohedrally shaped crystals were obtained after 4 days and were subsequented transferred to the crystallization solution supplemented to 30% (w/v) polyethylene glycol 2000 monomethyl ether before vitrification by direct immersion into liquid nitrogen.

X-ray diffraction (XRD) data were collected at the Cornell High Energy Synchrotron Source (Beam line ID7B2 MacCHESS, Ithaca, NY) using a Dectris Pilatus 6M detector system. Diffraction data were integrated and scaled using the XDS package as implemented in autoProc. Crystallographic phases were determined by the molecular replacement method as implemented in Phaser using an Alphafold model on the catalytic domain of CylA. Automated using wARP and manual model building using COOT, interspersed with rounds of crystallographic refinement using REFMAC5 resulted in the final model. Cross-validation, using 5% of the data for the calculation of the free R factor, was utilized throughout model building process in order to monitor building bias. The stereochemistry of all of the models was routinely monitored throughout the course of refinement using PROCHECK. Relevant data collection, phasing, and refinement statistics are provided in Moreira et al., Supplemental materials. The structure has been deposited at the Protein Databank as PDB ID: 9EKQ.

### Analytical Size Exclusion Chromatography.

Samples of His₆-CylA-27-412 prepared as described in ‘Expression and purification of His₆-CylA-27-412’ were diluted to a concentration of 50 μM and injected (20 μL injection volume) into an Agilent 1260 infinity analytical high-performance liquid chromatography system equipped with a Biozen™ 1.8 μm, 300×4.6 mm LC column pre-equilibriated with aSEC buffer (100 mM NaHPO₄, 200 mM NaCl, pH=7.4). A flow rate of 0.25 mL/min was used for all experiments. Retention time was correlated to protein size using a Gel Filtration Standard (Biorad) and samples of carbonic anhydrase isolated from bovine erythrocytes (Sigma Aldrich) and albumin isolated from bovine serum (Sigma Aldrich).

### Co-Operative Pore Formation Assay.

Pyranine encapsulated liposomes were prepared as described in Zhang et al., ACS Chem. Biol. 19, 357-369 (2024). mCylL_{L} and mCylL_{S} were prepared as described in Tang et al., J. Ind. Microbiol. Biotechnol. 46, 537-549 (2019). To a well in a black 96-well plate (200 μL) was added 90 μL high pH buffer (5 mM MES, 5 mM Tricine, 5 mM NaCl, pH 7.4) containing CylA (4 nM final concentration). After adding inhibitor, the mixture was incubated at room temperature for 10 min then freshly prepared (no more than 2 days old) pyranine encapsulated DOPC liposomes were added such that the total DOPC concentration was 80 μM. The pyranine fluorescence signal (excitation wavelength=460 nm, emission wavelength=510 nm) was monitored for 8 min at room temperature then a 1:1 mixture of mCylL_{S}:mCylL_{L} was added such that the total peptide concentration was 500 nM. Pyranine fluorescence was monitored for 30 min then the Triton X-100 concentration was adjusted to 0.1% and the fluorescence intensity was monitored for another 5 min. The resulting curves were max-min normalized yielding the fraction of permeabilized liposomes. The maximum fraction permeabilized achieved during the 30 min period in between adding cytolysin and adding Triton X-100 was used as a measure of enzyme activity. This experiment was performed in duplicate or triplicate for each inhibitor at each concentration. The data collected was subjected to a concatenate non-linear fit to a dose-response function using Origin 7 yielding EC₅₀ values. Errors presented are those calculated by Origin 7 during the fitting process.

### Determination of the Kₘ for the FRET Peptide.

To determine its Kₘ, FRET peptide (125 μM to 2 μM) was combined with His₆-CylA-27-412 (20 nM) in PBS (pH=7.4) at 37° C. and the EDANS fluorescence signal (excitation wavelength=336 nm, emission wavelength=490 nm) was monitored for 4 h. The slope of the linear region of each curve was used as a metric of initial velocity and the data was fit to the Hill form of the Michaelis-Menten model yielding Kₘ and the Hill coefficient (h). Experiments were performed in triplicate and concatenate fitting was done using Origin 7.

### Determining Initial Rates Using the FRET Peptide Substrate.

His₆-CylA-27-412 (20 nM) in PBS (pH=7.4) was combined with inhibitor or mCylL_{L}, mCylL_{S}, or a stoichiometric mixture of mCylL_{L}:mCylL_{S} at various concentrations in a 96 well plate and the plate was incubated at 25° C. for 10 min before adding the FRET peptide substrate (10 μM). The emission intensity of EDANS was measured over a 4 h period. The slope of the linear portion of the emission intensity as a function of time curve was calculated (vᵢ), divided by the slope of an uninhibited control (vₒ), and the ratio (vᵢ/vₒ) was plotted as function of total inhibitor concentration. Experiments were performed in triplicate at each inhibitor concentration. The data collected was subjected to a concatenate non-linear fit to a dose-response function using Origin 7 yielding IC₅₀ values. Errors presented are those calculated by Origin 7 during the fitting process.

Activity Assays with C+ Enterococcus faecalis.

Rabbit erythrocytes (Hemostat) were washed with cold, sterile PBS and diluted in BHI to a concentration of 1% v/v. A 3 mL overnight culture of E. faecalis ATCC 29212 was centrifuged (4000×g, 10 min) and the pellet was resuspended in 7 mL of BHI and the suspension was centrifuged (4000×g, 10 min). The pellet was resuspended in BHI to achieve an OD₆₀₀ of 1.4 and 50 μL of this suspension was added to a well containing 50 μL of inhibitor (400 μM). The inhibitor and the E. faecalis were incubated at room temperature for 10 min before adding 100 μL of 1% v/v rabbit erythrocytes in BHI. The inhibitor-free control well contained the same contration of bacteria and rabbit erythrocytes. The sterile negative control and sterile positive control contained 100 μL of BHI and 100 μL of 1% v/v rabbit erythrocytes. The plate was incubated at 37° C. without shaking until complete hemolysis was observed in the inhibitor free wells (usually 4 h). Triton X-100 was then added to the sterile positive control wells to a concentration of 0.03% and the plate was incubated for another 10 min. The plate was then placed on ice and centrifuged at 1000×g for 10 min to pellet all the remaining cellular material. The supernatant was diluted 10-fold in PBS and the absorbance at 415 nm was measured. For each condition, four wells were prepared and analyzed. To determine an EC₅₀ the same experiment was performed with the same controls but inhibitor concentrations equal to 200, 150, 125, 100, 75, 50, 25, and 10 μM were used. The data collected was subjected to a concatenate non-linear fit to a dose-response function using Origin 7 yielding EC₅₀ values. Errors presented are those calculated by Origin 7 during the fitting process.

### Growth Curve Assay.

To a 96-well plate, BHI media was added containing 200 μM of inhibitor 9. An overnight culture of E. faecalis ATCC 29212 was diluted to a concentration of 10⁶ CFU/mL and an equal volume of this culture was added to the well containing inhibitor. A growth control was prepared by combining the diluted bacterial cells with an equal volume of sterile BHI. A sterility control well was filled with sterile BHI to the same volume. The 96-well plate was transferred to a plate reader preheated to 37° C. and the plate was shaken continuously. The OD₆₀₀ of each well was measured over the course of 24 h. Uncorrected OD₆₀₀ values were plotted as a function of time.

Cytoxicity Assays with HeLa Cells.

Cell culturing and maintenance was performed in Dulbecco's Modified Eagle Medium (DMEM, Gibco) supplemented with 10% heat inactivated fetal bovine serum (FBS, Gibco). HeLa cells were seeded in 96 well plates (Nunc) using a seeding density of 5000 cells/well and allowed to adhere for 16 h at 37° C., 5% CO₂. The next day, cells were treated with compounds 2, 3, 5-9 in 2-fold dilutions, starting at 100 μM. Post-treatment, plates were incubated for 72 h at 37° C., 5% CO₂. Subsequently, the growth medium was removed, and the cells were washed once with pre-warmed phosphate buffered saline (PBS; pH=7.2). Next, 50 μL of DMEM-FBS media containing 1 mg/mL of 3-(4,5-dimethylthiazol-2-yl)-2,5-diphenyltetrazolium bromide (MTT) was added to the cells and the cells were incubated in the dark at 37° C., 5% CO₂ for 4 h. During this time, live cells reduced yellow tetrazolium salts to purple formazan crystals through NAD(P)H-dependent oxidoreductase activity. After 4 h, the medium was removed carefully and the insoluble formazan crystals were solubilized by incubating with 100 μL of dimethyl sulfoxide (DMSO) in the dark for 1 h with intermittent swirling. Finally, absorbance was recorded at 575 nm (See Moreira et al., Supplementary FIG. 11).

Detection of CylL_{S}″ from Extracts of E. faecalis.

An overnight culture of E. faecalis ATCC 29212 was diluted to an OD₆₀₀ equal to 2.0 in BHI then further diluted 1000-fold in BHI. Inhibitor 9 or vehicle was added immediately after dilution and the inoculated samples were incubated with shaking at 37° C. for 20 h. Following incubation, 400 μL portions of each sample were flash frozen and lyophilized. Each lyophilized sample was extracted with 400 μL of 90% methonal/10% water containing ¹⁵N,¹³C-labelled CylL_{S}″ (5.8 nM). Alternating rounds of water bath sonication and vortexing were used to homogenize the samples. Once homogenized, the samples were allowed to stand at room temperature for 1 h then were centrifuged at 16000 g for 10 min, incubated at −20° C. for 1 h, and centrifuged at 16000 g for 10 min. The supernatant was analyzed by LCMS using injection volumes of 5 μL. EIC corresponding to the exact masses of [M+2H]²⁺ and [M+Na+H]²⁺ were integrated and the peak areas were added together giving the total CylL_{S}″ and internal standard responses. The concentration of CylL_{S}″ was estimated from the response ratio multiplied by the concentration of the internal standard. Errors are the standard deviations between 4 trials consisting of two technical replicates for each of the two biological replicates.

Determining the Relative Amounts of cylL, cylS, and cylA in Total RNA Extracts from E. faecalis.

The amounts of cylL, cylS, and cylA transcripts relative to the amount of 16S rRNA were determined by RT-qPCR. Amplicons and primers used to quantify cylL, cylS and 16S rRNA. Overnight cultures of E. faecalis ATCC 29212 were prepared as described in ‘Detection of CylL_{S}″ from extracts of E. faecalis’. Upon inoculation, samples were treated with 100 μM of 9 or vehicle. The samples were incubated at 37° C. for 20 h, then cells were collected by centrifugation (5000 g for 10 min). The supernatant was removed and the cells pellets were re-suspended in Lysis buffer (10 mM TrisHCl, 3 mg/mL lysozyme, pH=7.8) at a concentration of 160 μL of Lysis buffer for 1 mL of overnight culture. The cells were incubated in Lysis buffer at room temperature for 15 min then RNA was extracted using a Monarch® Total RNA Miniprep Kit. Isolated RNA was immediately converted to cDNA using an iScript™ cDNA Synthesis Kit. cDNA was amplified using a SsoAdvanced Universal SYBR Green Supermix and a StepOnePlus Real-Time PCR System (Applied Biosystems). The expression level of each gene in each sample was determined by taking the difference between the C_{T} of the amplification curve for the gene of interest (cylL, cylS or cylA) and the C_{T} of the amplification curve for 16S rRNA, yielding ΔC_{T}. ΔΔC_{T} was determined by taking the difference between the ΔC_{T} of the inhibitor treated sample and the ΔC_{T} of the inhibitor-free control for each gene. Fold change in gene expression was calculature using the following equation:⁷²

Fold change in gene expression=2^{ΔΔCT}

Two technical replicates for two biological replicates were conducted.

TABLE 2
Analysis of the high-resolution masses
observed for compounds 1-10.

| Compound | | Theoretical | Observed | Δ |
|---|---|---|---|---|
| number | Ion | m/z | m/z | (ppm) |
| 1 | [M + H—H₂O]⁺ | 624.2796 | 624.2858 | 6.2 |
| 1 | [2M + H—3H₂O]⁺ | 1265.5623 | 1265.5639 | 1.6 |
| 2 | [M + H—H₂O]⁺ | 756.3734 | 756.3776 | 4.2 |
| 2 | [M + 2H—2H₂O]⁺ | 369.6851 | 369.6863 | 1.2 |
| 3 | [M + H—H₂O]⁺ | 612.3159 | 612.3236 | 7.7 |
| 3 | [2M + H—3H₂O]⁺ | 1205.6140 | 1205.6190 | 5.0 |
| 4 | [M + H—2H₂O]⁺ | 638.2952 | 638.2968 | 1.6 |
| 5 | [M + H—2H₂O]⁺ | 580.2897 | 580.3000 | 10.3 |
| 5 | [2M + H—3H₂O]⁺ | 1177.5827 | 1177.5924 | 9.7 |
| 6 | [M + H—2H₂O]⁺ | 638.2952 | 638.3034 | 8.2 |
| 6 | [M + H—3H₂O]⁺ | 1293.5937 | 1293.6039 | 10.2 |
| 7 | [M + H—2H₂O]⁺ | 610.263 | 610.2670 | 4.0 |
| 7 | [2M + H—3H₂O]⁺ | 1237.5311 | 1237.5351 | 4.0 |
| 8 | [M + H—2H₂O]⁺ | 654.2901 | 654.2959 | 5.8 |
| 8 | [M + H—4H₂O]⁺ | 1307.5759 | 1307.5729 | 3.0 |
| 9 | [M + H—2H₂O]⁺ | 622.2639 | 622.2678 | 3.9 |
| 9 | [2M + H—3H₂O]⁺ | 1261.5311 | 1261.5350 | 3.9 |
| 10 | [M + H]⁺ | 614.2781 | 614.2841 | 6.0 |

TABLE 3
IC₅₀ (μM) measurements of certain compounds.

| Compound number | IC50 (μM) |
|---|---|
| 11 | 3 ± 1 |
| 12 | 0.119 ± 0.023 |
| 13 | 10 ± 1 |
| 14 | 13 ± 1 |
| 15 | 3.3 ± 1.9 |
| 16 | 2.9 ± 0.8 |
| 17 | 7.5 ± 0.16 |
| 18 | 0.100 ± 0.013 |
| 19 | 0.043 ± 0.005 |
| 20 | 0.057 ± 0.006 |
| 21 | 17.8 ± 4 |
| 22 | 58 |
| 23 | 33.5 ± 13 |
| 24 | 37.6 ± 12 |

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A peptide comprising, from an N-terminus to a C-terminus:
Z-X¹-X²-X³-X⁴-X⁵-X⁶-BA,
wherein
Z is an acetyl group, a thiazole group, an imidazole group, a pyrazine group, a difluorophenyl group, a dichlorophenyl group, or absent, wherein Z, when present, is covalently bonded to an N-terminus nitrogen on X¹;
X¹ is Gly, Pro, Ser, cyclopropylglycine (Cpg), cyclopentylglycine (Cpg*), Asp, Gln, or Ala;
X² is Asp, Glu, Gln, Ala, Phe, Ile, Cpg, Norvaline (Nva), or 2-aminobutyric acid (Abu);
X³ is Lys, Gln, Val, Phe, Cpg, Ala, Pro, Nva, or absent;
X⁴ is Glu, Gln, Lys, Pro, Ala, Nva, or absent;
X⁵ is Ala, Pro, Nva, Abu, or absent; and
X⁶ is Gly, Glu, Nva, or absent; and
BA is a boronic acid moiety (—B(OH)₂) covalently bonded to a carbon atom of the amino acid at the C-terminus of the peptide or replaces the —(COOH) of the amino acid at the C-terminus of the peptide.

2. The peptide of claim 1, wherein the peptide comprises Z-X¹-X²-X³-X⁴-X⁵-X⁶ BA, and the peptide is one or more of:
i)
(SEQ ID NO: 1)
Ac-Gly-Asp-Val-Gln-Ala-Glu-BA;
ii)
(SEQ ID NO: 2)
Ac-Pro-Asp-Phe-Lys-Pro-Glu-BA;
iii)
(SEQ ID NO: 3)
Ac-Gly-Asp-Val-Gln-Ala-Nva-BA;
iv)
(SEQ ID NO: 4)
Ac-Gly-Glu-Val-Gln-Ala-Glu-BA;
v)
(SEQ ID NO: 5)
Ac-Gly-Abu-Val-Gln-Ala-Glu-BA;
vi)
(SEQ ID NO: 6)
Ac-Gly-Asp-Val-Gln-Abu-Glu-BA;
vii)
(SEQ ID NO: 7)
Ac-Gly-Asp-Val-Nva-Ala-Glu-BA;
viii)
(SEQ ID NO: 8)
Ac-Ser-Asp-Val-Gln-Ala-Glu-BA;
ix)
(SEQ ID NO: 9)
Ac-Gly-Asp-Cpg-Gln-Ala-Glu-BA;
and
x)
a salt or solvate thereof;
wherein Z is an acetyl group (Ac) covalently bonded to an N-terminus nitrogen on X¹ and the BA is covalently bonded to a carbon atom of X⁶ or replaces the —(COOH) of the X⁶.

3. The peptide of claim 1, wherein the peptide comprises Z-X¹-X²-X³-X⁴-X⁵-BA, and the peptide is one or more of:
xi) Ac-Asp-Phe-Lys-Pro-Nva-BA (SEQ ID NO: 10);
xii) Ac-Asp-Ile-Gln-Ala-Nva-BA (SEQ ID NO: 11);
xiii) Ac-Asp-Cpg-Gln-Ala-Nva-BA; and
xiv) a salt or solvate thereof;
wherein Z is an acetyl group (Ac) covalently bonded to an N-terminus nitrogen on X¹ and the BA is covalently bonded to a carbon atom of X⁵ or replaces the —(COOH) of the X⁵.

4. The peptide of claim 1, wherein the peptide comprises Z-X¹-X²-×3-X⁴-BA, and the peptide is one or more of:
xv)
thia-Cpg-Gln-Ala-Glu-BA;
xvi)
Ac-Cpg-Gln-Ala-Glu-BA;
xvii)
imi-Cpg-Gln-Ala-Glu-BA;
xviii)
pyr-Cpg-Gln-Ala-Glu-BA;
xix)
dif-Cpg-Gln-Ala-Glu-BA;
xx)
dic-Cpg-Gln-Ala-Glu-BA;
xxi)
Ac-Cpg-Gln-Ala-Nva-BA;
xxii)
Ac-Cpg-Ala-Pro-Nva-BA;
xxiii)
Ab-Cpg*- Ala-Pro-Nva-BA;
and
xxiv)
a salt or solvate thereof;
wherein Z is an acetyl group (Ac), a thiazole group (thia), an imidazole group (imi), a pyrazine group (pyr), a difluorophenyl group (dif), a dichlorophenyl group (dic), or absent (Ab), each covalently bonded to an N-terminus nitrogen on X¹ and the BA is covalently bonded to a carbon atom of X⁴ or replaces the —(COOH) of the X⁴.

5. The peptide of claim 1, wherein the peptide comprises Z-X¹-X²-X³-BA, and the peptide is one or more of:
XXV) Ac-Gln-Ala-Nva-BA; and
xxi) a salt or solvate thereof;
wherein Z is an acetyl group (Ac) covalently bonded to an N-terminus nitrogen on X¹ and the BA is covalently bonded to a carbon atom of X³ or replaces the —(COOH) of the X³.

6. The peptide of claim 1, wherein the peptide comprises Z-X¹-X²-BA, and the peptide is one or more of:
xxii) Ac-Pro-Nva-BA; and
xxiii) a salt or solvate thereof;
wherein Z is an acetyl group (Ac) covalently bonded to an N-terminus nitrogen on X¹ and the BA is covalently bonded to a carbon atom of X² or replaces the —(COOH) of the X².

7. A composition comprising the peptide of claim 1 and a pharmaceutically acceptable carrier, excipient, or diluent.

8. A method of treating an Enterococcus sp. infection comprising administering to a subject in need thereof a therapeutically effective amount of a peptide of claim 1, thereby treating the infection.

9. The method of claim 8, wherein the Enterococcus sp. infection comprises one or more of E. faecalis, E. faecium, E. avium, E. caccae, E. casseliflavus, E. dispar, E. durans, E. gallinarum, E. hirae, and E. raffinosus.

10. The method of claim 9, wherein the Enterococcus sp. infection is caused by one or both of E. faecalis and E. faecium.

11. An inhibitor of a CylA serine protease of Enterococcus faecalis, wherein the inhibitor is: 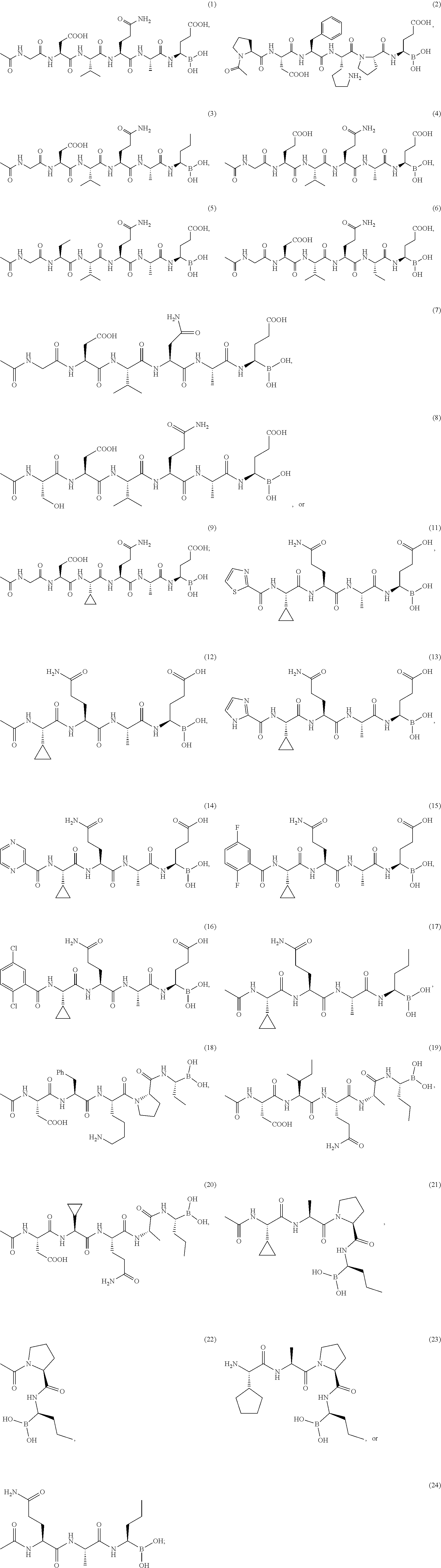
or a salt thereof.

12. The inhibitor of claim 11, wherein the inhibitor is: 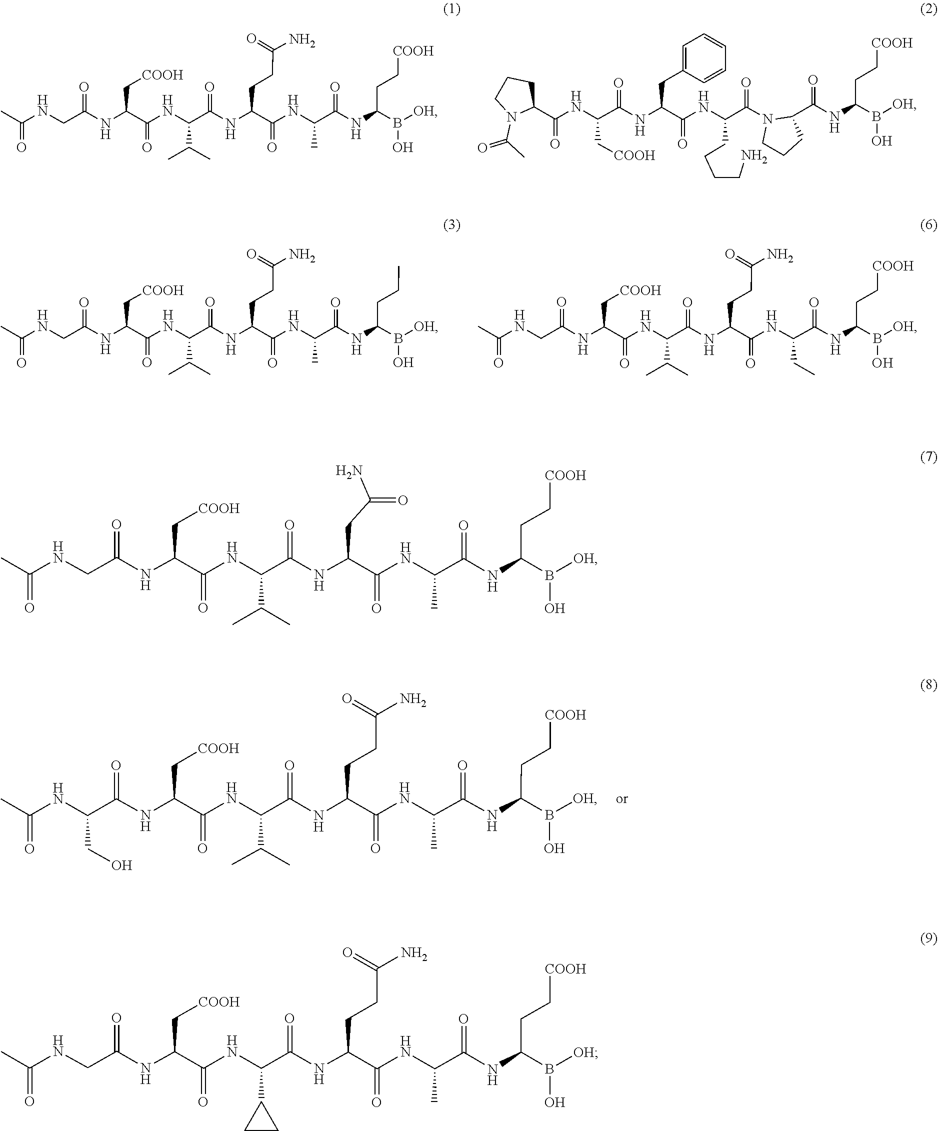
or a salt thereof.

13. A composition comprising the inhibitor of claim 11 and a pharmaceutically acceptable carrier, excipient, or diluent.


SEQUENCE LISTING (13 sequences)

SEQ ID NO: 1 - protein, 6 aa, Synthetic construct
  MOD_RES 6: note = Glu modifed to have boronic acid moiety replacing the carboxylic acid moiety
GDVQAX

SEQ ID NO: 2 - protein, 6 aa, Synthetic construct
  MOD_RES 6: note = Glu modifed to have boronic acid moiety replacing the carboxylic acid moiety
PDFKPX

SEQ ID NO: 3 - protein, 6 aa, Synthetic construct
  MOD_RES 6: note = Glu modifed to have boronic acid moiety replacing the carboxylic acid moiety
GDVQAX

SEQ ID NO: 4 - protein, 6 aa, Synthetic construct
  MOD_RES 6: note = Glu modifed to have boronic acid moiety replacing the carboxylic acid moiety
GEVQAX

SEQ ID NO: 5 - protein, 6 aa, Synthetic construct
  SITE 2: note = Abu
  MOD_RES 6: note = Glu modifed to have boronic acid moiety replacing the carboxylic acid moiety
GXVQAX

SEQ ID NO: 6 - protein, 6 aa, Synthetic construct
  SITE 5: note = Abu
  MOD_RES 6: note = Glu modifed to have boronic acid moiety replacing the carboxylic acid moiety
GDVQXX

SEQ ID NO: 7 - protein, 6 aa, Synthetic construct
  SITE 4: note = Nva
  MOD_RES 6: note = Glu modifed to have boronic acid moiety replacing the carboxylic acid moiety
GDVXAX

SEQ ID NO: 8 - protein, 6 aa, Synthetic construct
  MOD_RES 6: note = Glu modifed to have boronic acid moiety replacing the carboxylic acid moiety
SDVQAX

SEQ ID NO: 9 - protein, 6 aa, Synthetic construct
  SITE 3: note = Cyclopropylglycine
  MOD_RES 6: note = Glu modifed to have boronic acid moiety replacing the carboxylic acid moiety
GDXQAX

SEQ ID NO: 10 - protein, 5 aa, Synthetic construct
  MOD_RES 5: note = Nva modifed to replace the carboxlic acid with a boronic acid moiety
DFKPX

SEQ ID NO: 11 - protein, 5 aa, Synthetic construct
  MOD_RES 5: note = Nva modifed to replace the carboxlic acid with a boronic acid moiety
DIGAX

SEQ ID NO: 12 - protein, 6 aa, Synthetic construct
PDFKPE

SEQ ID NO: 13 - protein, 6 aa, Synthetic construct
GDVQAE